(19) United States
(12) Patent Application Publication
(10) Pub. No.: US 2020/0264194 A1
(43) Pub. Date: Aug. 20, 2020

(54) ANTIBODY VALIDATION USING IP-MASS SPECTROMETRY

(71) Applicant: Pierce Biotechnology, Inc., Carlsbad, CA (US)

(72) Inventors: John ROGERS, Rockton, IL (US); Bhavinkumar PATEL, Rockford, IL (US); Greg POTTS, Rockford, IL (US); Leigh FOSTER, Rockford, IL (US)

(21) Appl. No.: 16/305,589

(22) PCT Filed: May 26, 2017
(86) PCT No.: PCT/US2017/034634

§ 371 Date: Nov. 29, 2018

Related U.S. Application Data
(60) Provisional application No. 62/344,739, filed on Jun. 2, 2016.

(51) Int. Cl. G01N 33/68; G01N 33/541

(52) U.S. Cl. CPC G01N 33/6878; G01N 33/541; G01N 33/6848

(57) ABSTRACT

The invention relates, in part, to compositions and methods for validating antibodies utilizing immunoprecipitation and mass spectrometry.

### RELATED APPLICATIONS

This application is a 371 National Stage U.S. Nonprovisional Application of PCT/US2017/34634 filed on May 26, 2017, which claims the benefit of priority to U.S. Provisional Application No. 62/344,739, filed on Jun. 2, 2016 , which disclosures are herein incorporated by reference in their entireties.

### SEQUENCE LISTING

The instant application contains a Sequence Listing which has been submitted electronically in ASCII format and is hereby incorporated by reference in its entirety. Said ASCII copy, created on Jun. 7, 2017, is named LT01155PCT_SL.txt and is 532 bytes in size.

### FIELD OF THE INVENTION

The invention relates, in part, to compositions and methods for validating antibodies utilizing immunoprecipitation and mass spectrometry.

### BACKGROUND OF THE INVENTION

Antibodies are used in a broad range of research and diagnostic applications for the enrichment, detection, and quantitation of proteins and their modifications. Tens of thousands of antibodies are commercially available against thousands of proteins, which are used in a variety of applications, including Western blotting (WB), immunofluorescence (IF), immunoprecipitation (IP), flow cytometry (FC), chromatin IP (ChIP), enzyme linked immunoassays (ELISA), and bead-based sandwich assays (e.g. Luminex). These antibodies may be monoclonal or polyclonal from different organisms, and they may be used to interrogate biological systems and signaling pathways, diagnose disease, and assess responses to treatment (see Lipman, N.S. et al., ILAR Journal 46(3):258-268 (2005)).

Unfortunately, most antibodies are poorly characterized, both initially and between manufacturing lots. This is due to at least three challenges: 1) the sheer number of protein targets and antibodies can be overwhelming; 2) the unique requirements and challenges of each antibody target, application, and model system (such as native versus denatured epitope conformation or unstimulated vs. stimulated cells), and; 3) the lack of consistent standard approaches and criteria to assess antibody selectivity (see Madhusoodanan, J. Validating Antibodies: An Urgent Need. 2014; and Bordeaux, J. et al., Biotechniques 48(3):197-209 (2010)). There is a great need for improved antibody validation approaches and criteria to ensure that the reagents are fit-for-purpose. Multiple recommendations for antibody validation have been proposed, and databases of consolidated antibody annotation and performance “scoring” information are freely accessible (e.g., Antibodypedia, as described in Bjorling, E. and Uhlen M. Mol. Cell. Proteomics 7(10):2028-37 (2008)).

A variety of antibody validation criteria have been proposed, including: 1) the assessment of antibody specificity with genetic knockdowns or blocking peptides; 2) verification of antibody detection results with different biological systems (target localization, model systems, etc.); 3) correlation of antibody results between methods, and; 4) demonstration of reproducibility between samples, labs, and manufacturing lots (see Bordeaux, J. et al., Biotechniques 48(3):197-209 (2010), Bjorling, E. and Uhlen M. Mol. Cell. Proteomics 7(10):2028-37 (2008), and Pauly, D. and K. Hanack, [symbol] :691 (2015)).

Recently, mass spectrometric approaches have been proposed for antibody validation (see Bostrom et al., J Proteome Res 13(10):4424-35 (2014) and Marcon E. et al., Nature Methods 12:725-731 (2015)). Despite the cost and technical requirements of mass spectrometry, of all existing validation methods, mass spectrometry has the unique ability to identify the actual antibody target(s), isoforms, modifications, and associated proteins. No other method can identify and characterize an antibody target with the depth and specificity of MS. However, unlike Western blotting, ELISA, and other standard immunological methods that use blocking reagents, like milk or bovine serum albumin (BSA), to minimize background protein binding, mass spectrometry detects all proteins, specific and non-specific, in a sample. For example, immunoprecipitation with an antibody immobilized on a bead or resin is a common approach to enrich a target protein and associated proteins from a lysate or biofluid.

Due to the low abundance of a specific target relative to common background proteins, non-specifically bound proteins may overwhelm and interfere with or prevent the detection of a low abundance target when utilizing mass spectrometry. Even with stringent wash conditions and optimized reagents, dozens to hundreds of background proteins are typically observed in an immunoprecipitated sample that is analyzed by mass spectrometry. Thus, the mass spectrometry results from an immunoprecipitated sample can be difficult to filter and interpret. In particular, an assurance of antibody selectivity for a native protein from a real biological system is particularly challenging to demonstrate, in part because of the extremely low abundance of an intended target as compared to non-specific/background antibody binders.

To address these issues, we have developed a new approach to antibody validation. Through the use of optimized sample preparation reagents and methods, state of the art instrumentation, and a novel data analysis pipeline, a comprehensive workflow has been created to validate antibodies for their intended target using immunoprecipitation combined with mass spectrometry (IP-MS, as shown in FIG. 1). The benefits of this IP-MS approach include identification of the antibody target(s), isoforms, and modifications, quantitative assessment of antibody selectivity by calculating fold-enrichment of targets and off-targets, and the accurate identification and quantification of target protein modifications and interacting proteins.

### SUMMARY OF THE INVENTION

The invention relates, in part, to compositions and methods for validating antibodies utilizing immunoprecipitation and mass spectrometry.

In some embodiments, the invention includes method for identifying proteins that specifically bind to an antibody is encompassed, wherein the method comprises:

i) Selecting a test antibody;
ii) Preparing a cell lysate from a biological sample;
iii) Contacting the cell lysate with the test antibody, and immunoprecipitating the antibody and its protein binding partner(s);
iv) Analyzing the immunoprecipitated antibody and its protein binding partner(s) by mass spectrometry;
v) Determining the fold enrichment of the protein(s) bound to the test antibody as compared to the proteins in the cell lysate; and
vi) Identifying the proteins that specifically bind to the antibody, wherein proteins that specifically bind to the antibody are enriched as compared to proteins in the cell lysate.

In some aspects, the antibody binds to more than one target protein. In some embodiments, the antibody is characterized according to its specificity to its target proteins, wherein a larger fold enrichment, or greater signal intensity, for one target protein as compared to another target protein, means the antibody is more specific for that protein than for a protein with a smaller fold enrichment or lesser signal intensity.

In some embodiments, the method for identifying proteins that specifically bind to an antibody comprises:

i) Selecting a test antibody;
ii) Preparing a first and second preparation of cell lysate from a biological sample, wherein the first and second preparations are nearly identical;
iii) Contacting the first cell lysate with the test antibody, and the second cell lysate with a second antibody, and immunoprecipitating the antibodies and their protein binding partner(s);
iv) Analyzing the immunoprecipitated test and second antibody and their protein binding partner(s) by mass spectrometry;
v) Plotting the intensity and/or fold enrichment of each protein identified by mass spectrometry as being bound to the test antibody on an x- or y-axis, and plotting the intensity or fold enrichment of each protein identified by mass spectrometry as being bound to the second antibody on the opposite axis; and
vi) Identifying the proteins that specifically bind to the test and second antibodies, wherein

a. proteins that specifically bind to the test and second antibody are those that do not display equal or nearly equal binding to both the test and second antibodies (those that fall along the diagonal when plotted);

b. proteins that specifically bind to the test antibody fall above the diagonal if plotted along the y-axis, or below the diagonal if plotted along the x-axis; and

c. proteins that specifically bind to the second antibody fall above the diagonal if plotted along the y-axis, or below the diagonal if plotted along the x-axis.

In some aspects, the antibody binds to more than one target protein. In some embodiments the antibody is characterized according to its specificity to its target protein(s), wherein a larger fold enrichment, or greater signal intensity, for one target protein as compared to another target protein means the antibody is more specific for that protein than for a protein with a smaller fold enrichment or lesser signal intensity.

In some embodiments, the test and second antibody are the same, and protein in excess of what is needed to saturate the protein binding sites on the test antibody is added to the first cell lysate, but not the second cell lysate, prior to contact with the antibody. Proteins that specifically bind to the test and second antibody are those that do not display equal or nearly equal binding to both the test and second antibodies (those that fall along the diagonal when plotted).

The biological sample used in the methods may be a cell in cell culture, tissue, blood, serum, plasma, cerebral spinal fluid, urine, synovial fluid, peritoneal fluid, or other biofluids. The biological sample may be stimulated or activated prior to contact with antibody, and the stimulation may be with a growth factor, hormone, toxin, inhibitor, or other test molecule.

In some embodiments, the cell in cell culture is a primary or secondary cell, immortal cell, or stem cell. In some aspects, the cell is selected from A549, BT549, HCT116, HEK293, HeLa, HepG2, Hs578T, LNCaP, MCF7, NIH3T3, SKMEL5, or SR. In some embodiments, the cell is in the NCI60 panel.

In some method embodiments, the cell lysate is fractionated. Fractionation may reduce the complexity of the cell lysate allowing for more accurate detection during mass spectrometry. Fractionation may encompass reducing the complexity of the digested cell lysate based on separation by molecular weight, size, hydrophobicity, ion exchange binding, hydrophilic interactions, or affinity enrichment.

In some embodiments, fold enrichment is determined by the formula

$$fold enrichment = \frac{\frac{target protein abundance in the immunoprecipitate ( IP )}{total protein abundance in IP}}{\frac{target protein abundance in whole lysate}{total protein abundance in whole lysate}} ,$$

wherein a target protein is a protein bound to the test antibody.

In some embodiments, the protein(s) that specifically bind to the antibody are enriched about 5-fold or higher as compared to the protein(s) in the cell lysate. In some aspects, the fold enrichment is about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or greater than 200-fold higher as compared to the protein(s) in the cell lysate.

The second antibody used in methods comprising two antibodies may be i) an antibody that is believed to bind to a subset of the same protein or proteins as the test antibody; or an antibody that is not believed to bind to the same protein or proteins as the test antibody. In some aspects, the second antibody is an isoform-specific antibody or a pan-specific antibody.

In embodiments comprising plotting, the plot may be a scatter plot, and the intensity may be quantified. The quantification may be done label-free, or via metabolic or chemical mass tagging techniques. The quantification may measure peptide signal intensity, or use label free protein quantitation (LFQ), intensity-based absolute protein quantitation (iBAQ), spectral counts, sequence coverage, number of unique peptides, or protein rank, for example.

In some embodiments, the fold enrichment is determined and plotted.

In some embodiments, the identified proteins are further characterized by sequencing.

In some embodiments, the identified proteins are post translationally modified (PTM). Post translational modifications include, but are not limited to, phosphorylation, glycosylation, ubiquitination, nitrosylation, methylation, acetylation, lipidation and proteolysis.

In some embodiments, the identified interaction partners, isoforms, or modifications may indicate distinguishable epitopes for different antibodies.

In some embodiments, a method for determining the relative performance of more than one antibody is encompassed. In some embodiments, the methods involve comparing the performance of the test antibodies, or the test and second antibody, against each other, and ranking their performance based upon signal intensity, fold enrichment, sequence coverage, number of unique peptides, or spectral counts, wherein one antibody performs better than another with respect to a particular target protein if its signal intensity, fold enrichment, sequence coverage, number of unique peptides, or spectral counts is greater than with another antibody. The results of this comparison of relative antibody performance may indicate relative antibody affinity for the target.

In some embodiments, the mass spectrometry is tandem mass spectrometry, optionally using data dependent acquisition. In some embodiments, the mass spectrometry uses data independent acquisition.

In some aspects, the immunoprecipitated antibody-target protein is digested prior to mass spectrometry. The digesting may comprise a protease or chemical digest, and may be single or sequential.

In some embodiments, the protease digestion is with trypsin, chymotrypsin, AspN, GluC, LysC, LysN, ArgC, proteinase K, pepsin, clostripain, elastase, GluC bicarbonate, LysC/P, LysN promisc, protein endopeptidase, staph protease or thermolysin.

In some embodiments, the chemical cleavage is with CNBr, iodosobenzoate or formic acid.

In some aspects, the methods comprise desalting after immunoprecipitation or after digestion and prior to mass spectrometry.

The invention further includes compositions and method for characterizing an antibody (e.g., a high affinity antibody), the method comprising: (a) determining the affinity of the antibody to an antigen, and (b) determining the selectivity of the antibody for the antigen, wherein the affinity of the antibody and/or the selectivity of the antibody are determined using immunoprecipitation-mass spectrometry (IP-MS), and wherein the immunoprecipitate is generated by contacting the antigen with the antibody under conditions that allow for the formation on the immunoprecipitate between the antibody and the antigen. In some instances, selectivity of the antibody for its binding partner may be determined by the detection of binding to molecules (e.g., proteins) in a cell lysate. Further, the cell lysate may be derived from a cell of a species which expresses all of part of the antigen. Additionally, selectivity may determined by western blot of the cell lysate. Selectivity may be determined using cells which expresses all of part of the antigen from more than one species. Similarly, selectivity may be determined by western blot of cell lysates from more than one species. In some instances, selectivity may be determined by generating an immunoprecipitate of a cell extract using the antigen, followed by identification and/or quantification of two or more non-antibody molecules present in the immunoprecipitate. Further, the ratio of antigen/non-antibody molecules may be calculated in the immunoprecipitate.

The invention further includes methods for preparing a matched set of antibodies, as well as the matched sets themselves. Such methods may comprising: (a) determining the affinity of each antibody for its respective antigen in a cell lysate, (b) determining the selectivity of each antibody for its respective antigen in the cell lysate, and (c) selecting antibodies to form the matched set, wherein the affinity of the antibody and/or the selectivity of the antibody are determined using immunoprecipitation-mass spectrometry (IP-MS), and wherein the matched set is composed of two or more antibodies that each have selectivity of at least 100 fold (e.g., from about 100 fold to about 1,000, from about 200 fold to about 1,000, from about 300 fold to about 1,000, from about 400 fold to about 1,000, from about 5 fold to about 1,000, from about 100 fold to about 800, from about 100 fold to about 650, from about 100 fold to about 500, from about 200 fold to about 750, etc., fold) enrichment of for its respective antigen present in the cell lysate. In some instances, the two or more antibodies in a matched set may have affinities for their respective antigens with one log of each other. Further, the matched sets of the invention may contain from about two to about fifty (e.g., from about three to about ten, from about two to about thirty, from about two to about twenty, from about three to about fifteen, from about three to about forty, etc.) antibodies. In some instances, the antibodies of matched sets may bind to related target antigens. Further, the related targets antigens may be pre- and post-translationally modified forms of the same protein. In specific instances, the related targets antigens may be pre-translationally modified form of the protein unphosphorylated and post-translationally modified forms of phosphorylated protein.

In additional aspects, the invention includes methods for determining the selectivity of antibodies. In some instances, such methods comprise: (a) contacting the antibody with a cell extract under conditions that allow for the formation on an immunoprecipitate between the antibody and one or more antigen in the cell extract, (b) collecting the immunprecipitate formed in step (a), and (c) indentifying one or more non-antibody molecules present in the immunprecipitate by mass spectrometry, wherein the cell extract contains cell components from two or more cell types or one or more cell types from two or more species. In some instances, the two or more cell types may be from the same species. Further, the cell types may be obtained from two or more of the following tissues: (a) muscular, (b) connective, (c) nervous, and (d) epithelial. In specific instances, the connective tissue may be blood. Additionally, the two or more cell types may be from different species. Further, the two or more cell types from different species may be obtained from two or more tissues from each species.

The invention also includes methods for determining the selectivity of antibodies. Such methods may comprise: (a) contacting the antibody with two or more proteins under conditions that allow for the formation on an immunoprecipitate between the antibody and one or more of the two or more proteins, (b) collecting the immunprecipitate formed in step (a), and (c) quantifying the amount of individual proteins present in the immunprecipitate by mass spectrometry.

The invention also includes compositions comprising: (a) one or more cell extract obtained from one or more cell types, and (b) one or more exogenously added antibody, wherein the cell types are from two or more different species, as well as methods for using such compositions. Further, at least one of the cell extract may be prepared from cell lysates. Additionally, the cell lysates may obtained by lysing cells of the two or more cells types, followed by centrifugation (e.g., at greater or equal to 10,000×g for at least 15 minutes) of the resulting lysate to remove insoluble matter. Further, at least one of the antibodies may have affinity for at least one protein present in the cell extract.

The invention also includes methods for screening antibodies for binging acitivity to molecules other than a target molecule (e.g., target antigen). This may be done by the use of a cell extract from a cell that does not express the target molecule. By way of example, an antibody directed to a target protein may be screening using two cell extracts derived from cell lines that differ in expression of the target antigen. One of the cell lines may be known to express the target antigen and the other cell line may be believed or know to not express the antigen. By way of a more specific example, the gene encoding the target antigen may be disrupted or suppressed in one cell line. Suppression of expression may be performed through the usee of RNAi. Disruption of the gene may be performed by gene diting technologies (e.g., homologous recombination, zinc finger-FokI nucleases, CRISPR nucleases, TAL nucleases, etc.). Thus, the invention includes compositions and methods for identifying antibodies with high levels of binding specificity for a target molecules, as well as antibodies identified by such methods.

The invention also includes methods for identifying antibodies that selectively binds to target molecules of cells obtained from different species. Such methods may comprise: (a) contacting the antibody with two or more cell lysates generated from cells of different species under conditions that allow for the formation on two or more immunoprecipitates between the antibody and one or more target molecule present in each cell lysate, (b) collecting the immunoprecipitate from each cell lysate, and (c) determining the fold purification for the target molecules in each immunoprecipitate by mass spectrometry. Such cell may be derived from one or more species are selected from the group consisting of: (a) Homo sapiens, (b) Oryctolagus cuniculus, (c) Mus musculus, and (d) Rattus norvegicus. Further, antibodies used in the aspect of the invention may be generated in response to an epitope or a protein that is conserved across the different species from which the cell lysates are obtained. Additionally, the epitope may be from a protein in a category selected from the group consisting of: (a) heat shock proteins, (b) polymerases, (c) cell surface receptors, (d) transcription factors, (e) kinases, (f) dephosphorylases, (g) membrane associated transporters, and (h) zinc finger proteins.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIG. 1 shows an experimental workflow for antibody validation by immunoprecipitation with mass spectrometric analysis (IP-MS).

FIG. 2 presents a Venn diagram of the number of proteins identified from HepG2, A549, MCF7, BT549, and LNCaP cells of the NCI60 cell line panel by deep mass spectrometric analysis.

FIGS. 3A-3H provide protein identification and quantification across cell lines. FIG. 3A shows comparison of E-cadherin (CDH1) across twelve cell lines in unfractionated samples, while FIG. 3B shows comparison of CDH1 in fractionated samples for deeper proteome analysis. FIG. 3C shows N-cadherin (CDH2) protein expression across twelve cell lines in unfractionated samples, while FIG. 3D shows comparison of CDH2 in fractionated samples for deeper proteome analysis. FIGS. 3E-3H show distribution of expressed proteins detected in unfractionated MCF7 samples (FIG. 3E), fractionated MCF7 samples (FIG. 3F), in unfractionated A549 samples (FIG. 3G), and in fractionated A549 samples (FIG. 3H). In FIGS. 3E-3H, the expression levels of CDH1 and CHD2 are highlighted with an arrow indicating the expression level of the target protein(s).

FIGS. 4A-4B provide a comparison of antibodies immunoprecipitating two targets. In FIG. 4A, p53 protein was immunoprecipitated from BT549 cell lysate with the indicated antibodies on multiple days and quantified using the MS intensity of the three most intense peptides. In FIG. 4B, CDH1 was immunoprecipitated from MCF7 cell lysate with the indicated antibodies on multiple days and quantified using the number of identified peptides (y-axis and line), and by the calculated fold-enrichment of CDH1 from unfractionated and fractionated MCF7 cell lysates using label free quantitation (LFQ) values (vertical bars). Asterisks highlight the antibodies annotated as IP validated by IP/Western blot.

FIGS. 5A-5G provide information on filtering and visualization of specific proteins captured and quantified by IP-MS. FIG. 5A shows a scatterplot of the clusters of proteins quantified after immunoprecipitation with positive and negative control antibodies. “Negative control” indicates that the abundance of a protein was increased by a control IP (i.e., IP with a non-specific antibody). “Background” indicates that the abundance of a protein was increased in a similar manner for both a control IP (i.e., IP with a non-specific antibody) and a target IP (i.e., IP with a target-specific antibody). “Positive control” indicates that the abundance of a protein was increased with a target IP, while the abundance of this same protein was not increased by the control IP. FIG. 5B shows scatterplot results of the proteins captured by positive and negative control anti-CDH1 antibodies and quantified with MS. Fold-enrichment results relative to MCF7 lysates using iBAQ quantitation are colored. FIG. 5C shows fold-enrichment data based on iBAQ analysis following IP with anti-CDH1 antibody. FIG. 5D shows the interaction analysis from STRING for specifically captured proteins enriched >50-fold. FIG. 5E shows fold-enrichment of cadherin targets and additional proteins from A549 cell lysate with pan anti-cadherin antibody PA1-37199 compared with the pan anti-cadherin antibody PA5-16481 that does not immunoprecipitate, along with annotation of known interactors. FIG. 5F shows the interaction diagram from STRING database highlighting enriched proteins following IP with PA2-37199. FIG. 5G shows analysis of Gene Ontology (GO) term enrichment based upon the list of specifically enriched proteins.

FIGS. 6A-6D provide a comparison of several antibodies ability to immunoprecipitate CDKN1A. In FIG. 6A, CDKN1A protein was immunoprecipitated from HCT116 cell lysate with the indicated antibodies, and quantified using the MS intensity of the three most intense peptides. FIG. 6B shows enrichment of CDKN1A target and additional proteins with antibody PA1-30399 and annotation of known interactors. FIG. 6C shows the interaction diagram from STRING database highlighting enriched proteins following IP with PA1-30399. FIG. 6D shows analysis of Gene Ontology (GO) term enrichment based upon the list of specifically enriched proteins.

FIGS. 7A and 7B present MS data using a variety of ERBB2-specific antibodies. FIG. 7A presents fold-enrichment data with various antibodies following anti-ERBB2 immunoprecipitations versus unfractionated samples using MaxQuant quantitative analysis software (Thermo Fisher). FIG. 7B presents the number of ERBB2 peptides determined using MaxQuant quantitative analysis software versus Proteome Discoverer (PD1.4, Thermo Fisher) software. Numbers to the right of each bar indicate exact values. Pos Ctl=positive control.

FIGS. 8A, 8B, and 8C present fold-enrichment results after using a variety of CTNNB1-specific antibodies. FIG. 8A presents fold-enrichment data with various antibodies following anti-CTNNB1 immunoprecipitations versus fractionated samples using MaxQuant quantitative analysis software (Max Planck Institute). FIG. 8B presents the network interaction diagram of known interactors of CTNNB1 from the STRING database. FIG. 8C presents the two-dimensional hierarchical clustering result of the number of peptides for each protein interactor enriched with each anti-CTNNB1 antibody.

FIGS. 9A and 9B present the fold-enrichment of NFKB1A and its interaction partners. FIG. 9A shows the fold-enrichment of NFKB1A and multiple interaction partners from a LNCAP cell lysate. FIG. 9B shows the STRING database network interaction diagram for the proteins detected by IP-MS after immunocapture of NFKB1A (circled).

FIGS. 10A and 10B present the detection of contaminating peptide antigens in purified antibodies. FIG. 10A shows the level of target protein in neat antibody preparations that were mixed with bovine serum albumin prior to IP-MS analysis. FIG. 10B shows the light and heavy peptide signal intensities for the three most intense peptide signals enriched from HEK293 cells grown in heavy lysine(+8 Da) and arginine(+10 Da) isotope-labeled amino acids.

### DESCRIPTION OF THE EMBODIMENTS

### Definitions

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

As used herein “protein”, “peptide”, and “polypeptide” are used interchangeably throughout to mean a chain of amino acids wherein each amino acid is connected to the next by a peptide bond. In some embodiments, when a chain of amino acids consists of about two to forty amino acids, the term “peptide” is used. However, the term “peptide” should not be considered limiting unless expressly indicated.

The term “antibody” is used in the broadest sense and encompasses various antibody structures, including but not limited to monoclonal antibodies, polyclonal antibodies, multispecific antibodies (such as bispecific antibodies), and antibody fragments so long as they exhibit the desired immunoprecipitating activity. As such, the term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment. Pepsin treatment yields a F(ab′)₂ fragment that has two antigen-binding sites. The term antibody also includes, but is not limited to, chimeric antibodies, humanized antibodies, and antibodies of various species such as mouse, goat, horse, sheep, chicken, etc. Furthermore, for all antibody constructs provided herein, variants having the sequences from other organisms are also contemplated, such as CDR-grafted antibodies or chimeric antibodies. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct. The antibodies are referred to by reference to name and catalog reference. The skilled artisan, holding this name and catalog information, is capable of determining the sequence of the antibody, and therefore the methods encompass any antibody having at least partial sequence of a reference antibody so long as the antibody maintains its ability to immunoprecipitate its antigen protein.

Mass spectrometry (MS) is a primary technique for analysis of proteins on the basis of their mass-to-charge ratio (m/z). MS techniques generally include ionization of compounds and optional fragmentation of the resulting ions, as well as detection and analysis of the m/z of the ions and/or fragment ions followed by calculation of corresponding ionic masses. A “mass spectrometer” generally includes an ionizer and an ion detector. “Mass spectrometry,” “mass spec,” “mass spectroscopy,” and “MS” are used interchangeably throughout.

“Targeted mass spectrometry,” also referred to herein as “targeted mass spec,” “targeted MS,” and “tMS” increases the speed, sensitivity, and quantitative precision of mass spec analysis. Non-targeted mass spectrometry, sometimes referred to as “data-dependent scanning,” “discovery MS,” and “dMS” and targeted mass spec are alike in that in each, analytes (proteins, small molecules, or peptides) are infused or eluted from a reversed phase column attached to a liquid chromatography instrument and converted to gas phase ions by electrospray ionization. Analytes are fragmented in the mass spec (a process known as tandem MS or MS/MS), and fragment and parent masses are used to establish the identity of the analyte. Peptide fragmentation for discovery MS can be triggered based upon the intensity of eluting peptides in a data-dependent manner (DDA), or fragmentation can be programmed to occur by isolation and fragmentation of peptide ions one or more mass ranges in a data-indepedent manner (DIA), such as by scanning, isolating, and fragmenting m/z windows across the MS1 spectra. Discovery MS analyzes the entire content of the MS/MS fragmentation spectrum. In contrast, in targeted mass spectrometry, a reference spectrum is used to guide analysis to only a few selected fragment ions rather than the entire content.

### Overview

The invention relates, in part, to compositions and methods for validating antibodies utilizing immunoprecipitation and mass spectrometry. Pursuant to the invention, antibodies may be analyzed alone or in comparion to other antibodies. Further, antibodies may be characterized by the affinity and/or specificity for a particular antigen and/or epitope. One goal of the invention is to identify antibodies that have particular characteristics that rendered them useful in more than one (e.g., two, three, four, five, etc.) antibody based methods (e.g., immunofluorescence, western blot, etc.). A further goal is the identification of antibodies that have enhanced suitability over their peers in one or more application (e.g., an ELISA).

The methods disclosed herein may be applied to any type of MS analysis. The methods are not limited by the specific equipment or analysis used. The use of any equipment with the intent of analyzing the m/z of a sample would be included in the definition of mass spectrometry. Non-limiting examples of MS analysis and/or equipment that may be used include electrospray ionization, ion mobility, time-of-flight, tandem, ion trap, DDA, DIA, and Orbitrap. The methods are neither limited by the type of ionizer or detector used in the MS analysis nor by the specific configuration of the MS. The methods are not limited to use with specific equipment or software. The methods are not limited to the equipment and software described in the Examples.

The invention relates, in part, to compositions and methods for assessing antibody affinity and specificity for their cogenate ligands (e.g., a protein). In some aspects, the invention include ligand quantification in immunoprecipitation samples. The invention also includes the comparison of the amounts of ligands obtained by the immunoprecipitation of different samples. In such instances, the comparison is often by comparing the amount of ligand present in two immunoprecipitation samples, resulting in the determination of fold enrichment. Further, in some instances, a “benchmark” may be determined, to which other samples are compared. For example, three samples may be compared to each other, where the amount of protein bound by each of three different antibodies is assessed. Thus, in this example, a single sample (e.g., a cell lysate) may be split into three aliquots. Different antibodies know to bind the protein may be added to each aliquot under conditions that allow for the formation of immunoprecipitates. The amounts of the protein present in the three immunoprecipitates may then be measured and compared to each other. The benchmark in such an instances may be any of the three antibodies or it may be, for example, the antibody that generates an immunoprecipitate with the least amount of protein in it.

A more specific example is as follows. Assume that the ligand is the p53 protein and that the p53 protein is present in a HeLa cell lysate (e.g., the cells are lysed and insoluble material is pelleted at 10,000×g, for 5 minutes at 4° C.). The lysate is split into three aliquots and Antibodies 1, 2, and 3 are added to each aliquot to generate an immunoprecipitate. It is then determined by MS that 6.2 μg, 1.2 μg, and 9.5 μg of p53 is present, respectively, in each of the aliquots to which Antibodies 1, 2, and 3 were added. If Antibody 2 is used as a benchmark, then Antibody 1 yields a 5.2 fold enrichment and Antibody 3 yields a 7.9 fold enrichment. Thus, the invention includes method for comparing antibodies to each other by one or more functional characteristics. In this example, the functional characteristic is the quantity of antigen that the antibodies precipitate.

Further, multiple replicates may be performed to generate statistical data for assessing and comparing antibody characteristics. Additionally, replicates may be generated using the same sample (e.g., the same HeLa cell lysate) or different samples (e.g., lysate made form different HeLa cell cultures). Using the above example as a point of reference, three different lysates may be tested with all three antibodies under conditions that allow for the formation of immunoprecipitates, with the average of the amount of p53 protein being used to determine fold enrichment.

The invention thus includes compositions and methods for comparing two or more antibodies to each other by IP-MS. This comparison will often be directed to the affinity and specificity of the antibodies being compared.

One test system for assessing antibody affinity may involve the use of purified ligands (e.g., proteins). As an example, immunoprecipitation reactions may be set up using multiple aliquots of a purified protein and identical amounts of different antibodies known to bind the protein may be added to each aliquot. The immunoprecipitate may then be analyzed by MS to determine the amount of protein present and/or the region of the protein to which the various antibodies are bound. Such a comparison would yield data related to affinity but little data related to specificity. One indication of specificity would be bioinformatic in that indentification of the regions of the protein to which the antibodies bind allows for the searched of proteins sequence databases for the identification of proteins with similar or identical amino acid sequences, as well as organisms that contain such proteins and cells that expresss such proteins.

The invention thus includes, in part, methods for comparing the binding characteristics of different antibodies to a purifified protein. Such comparisons can be used to determine the comparative affinity of the various antibodies to antigens.

Methods of the invention may also be used to measure the ability of antibodies to precipitate antigens from a mixture of antigens either individually or in comparison to other antibodies. As an example, a target antigen may be mixed with similar molecules to measure the ability of the antibody(ies) to distinguish the target antigen from the related molecules. One example would be where a human protein in the target antigen. The human protein may be mixed with the corresponding mouse protein or the corresponding mouse and rat proteins. A more specific example is as follows. Homo sapiens (human) p53 is a 393 amino acid protein and Mus musculus (mouse) p53 is a 381 amino acid protein. Further, these two proteins share about 71% amino acid sequence identity, with much of the sequence identity being in the central portion of the two proteins.

An antibody may be generated in response to human p53 protein and the ability of the antibody to distinguish between the human mand mouse forms of this protein may be assessed by mixing the two proteins and measuring the ability of the antibody to precipitate the human p53 protein. Assume that the two proteins are mixed in a 1:1 ratio and and the antibody generated in response to human p53 protein precipitates both proteins equally. In other words, when a mixture of 1:1 human and mouse p53 protein is contacted with an antibody generated in response to to human p53 protein, then ratio of human:mouse p53 protein precipitated is a meaure of the differential affinity and/or specificity of the antibody towards the two proteins. For example, if the ratio of p53 proteins in the precipitate is 2:1, then the antibody has some specificity for mouse p53 protein but has a 2 fold higher affinity for human p53 protein. The invention thus includes compositions and methods for assessing target antigen specificity for antiugens that have regions of similar amino acid sequence and/or conformation.

The invention includes compositions and methods for comparing the ability of an antibody to distinguish between related antigens and between two or more antibodies to bind to a single antigen and to distinguish between two or more related antigens. For example, an antibody generated in response to a particular antigen, or subportion thereof (e.g., an epitope), is contacted with more than one related antigens, followed by immunoprecipitation. The amounts of the related antigens present in the precipitate are then determined and compared to each other. From this the ability of the antibody to distinguish between the two antigens may be determined. The antigen and related antigens may be combined in equal proportions or in non-equal proportions. Non-equal propertions may be used when, for example, the antibody preciptates significantly more of one antigen over another (e. g., from about 5 times to about 50 times, from about 10 times to about 50 times, from about 15 times to about 50 times, from about 5 times to about 40 times, from about 10 time 2 to about 40 times, etc.).

In instances where an antibody has substantially higher affinity for one antigen over another antigen, test solutions may be prepared to equilibrate the differential affinities. For example, if an antibody precipitates 10 times more of a first antigen as compared to a second antigen, then the test solution may contain 10 times more of the second antigen as compared to the first antigen. In such instances, an immunoprecipitate would be expected to contain roughly the same amounts of both antigens. This type of method allows for the “fine tuning” of antibody affinity and/or specificity characterization. Thus, the invention include compositions and method for comparing two or more antibodies with respect to affinity and/or specificity for a target antigen.

In some embodiments, the immunoprecipitated proteins may be reduced and alkylated prior to fragmentation (e.g., digestion). Samples that have been reduced and alkylated may comprises modifications, such as to cysteine residues (e.g., CAM).

The samples may optionally be desalted prior to analysis by mass spectrometry. Both enzymatic and chemical digestion is encompassed. Enzymatic digestion includes, but is not limited to, digestion with a protease such as, for example, trypsin, chymotrypsin, AspN, GluC, LysC, LysN, ArgC, proteinase K, pepsin, Clostripain, Elastase, GluC biocarb, LysC/P, LysN, Protein Endopeptidase, Staph Protease or thermolysin. Chemical digestion includes use of, for example, CNBr, iodosobenzoate and formic acid.

In some embodiments, the fragmentation protocol uses MS-grade commercially available proteases. Examples of proteases that may be used to digest samples include trypsin, endoproteinase GluC, endoproteinase ArgC, pepsin, chymotrypsin, LysN protease, LysC protease, GluC protease, AspN protease, proteinase K, and thermolysin. In some embodiments, a mixture of different proteases are used and the individual results are combined together after the digestion and analysis. In some embodiments, the digestion is incomplete in order to see larger, overlapping peptides. In some embodiments, the antibody digestion is performed with IdeS, IdeZ, pepsin, or papain to generate large antibody domains for “middle-down” protein characterization. In some embodiments, the fragmentation protocol uses trypsin that is modified. In some embodiments, a protein:protease ratio (w/w) of 10:1, 20:1, 25:1, 50:1, 66:1, or 100:1 may be used. In some embodiments, the trypsin used is at a concentration of about 100 ng/ml-1 mg/ml, or about 100 ng/ml-500 μg/ml, or about 100 ng/ml-100 μg/ml, or about 1 μg/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the digestion step is for 10 minutes to 48 hours, or 30 minutes to 48 hours, or 30 minutes to 24 hours, or 30 minutes to 16 hours, or 1 hour to 48 hours, or 1 hour to 24 hours, or 1 hour to 16 hours, or 1 to 8 hours, or 1 to 6 hours, or 1 to 4 hours. In some embodiments, the digestion step is incubated at a temperature between 20° C. and 45° C., or between 20° C. and 40° C., or between 22° C. and 40° C., or between 25° C. and 37° C. In some embodiments, the digestion step is incubated at 37° C. or 30° C. In some embodiments, a step is included to end the digestion step. The step to end the digestion protocol may be addition of a stop solution or a step of spinning or pelleting of a sample. In some embodiments, the digestion is followed by guanidation.

In some embodiments, the fragmentation protocol includes use of protein gels. In some embodiments, the fragmentation protocol comprises in-gel digestion. An exemplary commercially available kit for performing in-gel digestion is the In-Gel Tryptic Digestion Kit (Thermo Fisher Cat #89871).

In some embodiments, the fragmentation protocol is carried out in solution. An exemplary commercially available kit for performing in-solution digestion is the In-Solution Tryptic Digestion and Guanidiation Kit (Thermo Fisher Cat. No. 89895).

In some embodiments, the fragmentation protocol uses beads. In some embodiments, the fragmentation protocol comprises on-bead digestion. In some embodiments, agarose beads or Protein G beads are used. In some embodiments, magnetic beads are used.

In some embodiments, protein samples are separated using liquid chromatography before MS analysis. In some embodiments, fragmented samples are separated using liquid chromatography before MS analysis.

In some embodiments, the eluted intact proteins are analyzed directly by MS to identify intact masses and fragmentation products for intact protein identification and characterization.

In some embodiments, known amounts of isotope-labeled (e.g., heavy isotope-labeled) versions of control proteins and/or peptides can be used as internal standards for absolute quantitation and normalization of capture and digestion efficiency.

The invention also includes compositions and methods for identifying molecules that interact with other molecules. These molecules may be of various types, including chemical entities and biological molecules. As an example, digoxin is a chemical entity which can be bound by antibodies. For instance, digoxin may be introduced into a cell, then cellular contents may be exposed to and anti-digoxin antibody and molecules present within the cell may be identified. Along these lines, the invention further includes methods for identifying antibodies suited for such application using, for example, IP-MS. The invention thus includes compositions and methods for detecting interactome and proteome interactions and includes interactions with molecules not normally produced within a particular cell type (e.g., exogenously introduced chemical entities).

In some instances, the invention does not include compositions and methods for detecting interactome and proteome interactions in one or both of the following pathways: The AKT-mTOR Pathway and/or the Ras pathway. In some instances, the invention does not include compositions and methods for detecting interactome and proteome interactions related to one or more proteins set out in PCT/US2017/022062, filed Mar. 13, 2017; U.S. Provisional Application 62/308,051, filed Mar. 14, 2016; and U.S. Provisional Application 62/465,102, filed Feb. 28, 2017.

Cellular interactions (e.g., intracellular interactions) are known to change with cellular conditions. Examples of such cellular conditions include thermal environment (e.g., “heat shock”), stage of cell cycle, cell surface receptor stimulation, cellular mutations/alterations (e.g., disease states), etc. The invention thus includes compositions and methods for detecting cellular interactions, as well as compositions and methods for the selection of antibodies suited for detecting cellular interactions.

By way of example, the eukaryotic chaperonin TRiC/CCT is a hetero-oligomeric complex involved in protein folding and is estimated to interaction with as much as 10% of cytosolic proteins (Lopez et al., “The mechanism and function of group II chaperonins,” J. Mol. Biol., 427:2919-2930 (2015). A number of the proteins believed to associate with the TRiC/CCT complex are subject to cell cycle regulated expression. Further, TRiC/CCT complex alterations (e.g., mutations) are believed to be associated with a number of disease states, including Huntington's Disease.

As an example, CDC20 is a component of the anaphase promoting complex that is believed to be under different types of cell cycle regulation, including cell cycle expression, proteolysis and phosphorylation. Further, this protein is believed to associate with the TRiC/CCT complex.

Using the above by way of example, the invention includes compositions and methods for identifying intracellular interactions between molecules and molecular complexes. In some instances, such methods comprise (1) contacting a cell lysate with an antibody to a cellular component of interest, under conditions that allow for the formation of an immunoprecipitate between the antibody and the cellular component of interest and (2) analyzing the immunoprecipitate to identify one or more cellular components present therein. In many instances, one of the two or more cellular components will be the cellular component of interest. Further, in many instances, analysis may be performed by mass spectrometry and/or the antibody used will be identified as suitable for the application by mass spectrometry, for example, using methods set out herein.

A number of types of experiments fall within the scope of the invention. For example, in some experiments, two cellular samples are compared for the presence of molecular interactors. By way of specific example, an antibody with specificity for TRiC may be contacted with cellular lysates derived from two different cell types. These cell types may be brain tissue cells (e.g., from the substantia nigra) from an individual afflicted with Huntington's Disease to identify proteins and other cellular molecules that interact with the TRiC/CCT complex. Comparisons may then been made between which interactors and the amount of individual interactors present in the different samples. Thus, qualitative and quantitative comparative studies may be performed on different samples. Such comparative studies may be useful, for example, for identifying interactions associated with disease states and diagnostic methods.

Another type of experiment that may be conducted within the scope of the invention is one where cells of the same type are studied. One example of this is a cell cycle study where a control cell lysate is compared to one or more test cell lysates. As an example, digoxin is a chemical entity that bind to a sodium potassium adenosine triphosphatase (Na+/K+ ATPase), a protein complex present in a number of tissues, including the myocardium. Cardiac cells may be contacted with digoxin, then contacted with an anti-digoxin antibody, followed by the identification and quantification of cellular molecules present in immunoprecipitates. Further, the cardiac cells may be contacted with additional chemical entities, followed by analysis to compare whether the amount of total Na+/K+ ATPase complex, individual components of the complex, or other proteins that immunoprecipitated with the complex are increased, decreased, or remain the same. In some instances, it may be seen that one or more biological molecules may precipitate in an increased or the same amount, while one or more other biological molecules may precipitate in a lower amount. This effect may be seen when the cells are contacted with an additional chemical entity that either (1) enhances or (2) interferes or disrupts protein-protein interactions.

Another type of experiment that may be performed is where protein interactors are identified based upon cell type, development stage, or cell cycle. Using cell cycle for purposes of illustration, cell samples may be generated where the cells are synchronized. Extracts may then be generated from these cells, followed by analysis according to methods of the invention. In specific aspects, cells may be synchronized in two or more different parts of the cells cycle (e.g., G1, S, G2, or M), upon which cell lysates will be generated from synchronized cells Immunoprecipitates maybe then be generated from the cell lysates using, for example, an antibody with specificity for TRiC. Proteins then present in the precipitate may then be identified using, for example, mass spectrometry. Further, qualitative and quantitative measurements of the proteins present in the precipitate may then be used to interactors that differ during different phases of the cell cycle. Similar additional experiments may be designed where samples derived from more than two cells types analyzed. As an example, cellular lysates from cells synchronized in G1, S, G2, and M, as well as unsynchronized cells may each be generated, followed by the generation of immunoprecipitates using an antibody with specificity for TRiC. Qualitative and quantitative measurements of the proteins present in the precipitates may then be made and compared to identify differential interactions through the cell cycle.

Further, “multiparameter” interactions may also be identified. One example of a “multiparameter” interaction experiment is where two antibodies with specificity for different proteins believed to interact with each other are employed. For purposes of illustration, antibodies with specificity for TRiC and CDC20 may be employed, where immunopreciptate samples may be generated using each antibody separately and together. Qualitative and quantitative measurements of the proteins present in the precipitates may then be made and compared.

### Modified Proteins and Peptides

In some aspects, the invention relates to the characterization and use of antibodies for the with affinity and/or specificity for post-translationally modified proteins and peptides, as well as proteins and peptides that are not post-translationally modified at the locus or loci of interest. In many instances, such antibodies will have significant specificity for a post-translational modification site present in a specific protein. This is so because in many instances it will be desirable to use antibodies that are capable of binding with high affinity and specificity to a target epitope present in a cell or a cellular extract.

Post-translational modifications include, but are not limited to, phosphorylation, glycosylation, ubiquitination, nitrosylation, methylation, acetylation, and lipidation.

By way of example, the mouse Sox9 protein is believed to be phosphorylated in chrondrocytes at a serine residue located at position 211 by a process involving TGF-β (Coricor and Serra, Sci. Rep. 6, 38616; doi: 10.1038/srep38616 (2016)). Twenty-one amino acids of this phosphorylation site, with serine 211 as the center, is as follows: NAIFKALQAD S PHSSSGMSEV (SEQ ID NO: 1).

Using the Sox9 region referred to above, the invention includes methods involving one or more of the following steps:
1. The identification of suitable antigens (e.g., peptides) for the generation of potentially antibodies with affinity and/or specificity for phosphorylated and/or unphosphorylated forms of the antigen.
2. The generation and standard (non-MS) screening of one or more of antibodies directed to one or more of such antigens.
3. Testing and/or comparing of one or more of the resulting antibodies using IP-MS to identify antibodies with high levels of affinity and/or specificity for the desired antigen (phosphorylated or unphosphorylated).

Using the above twenty-one Sox9 amino acid sequence for purposes of illustration, one starting point in determining suitability of the peptide of sub-portions thereof for antibody generation can be to search sequence databases for related sequences. An amino acid sequence database search indicated that this twenty-one Sox9 amino acid sequence is highly conserved across mammalian species. Further, a Homo sapiens specific protein search suggested that this amino acid sequence is found only in Sox9 and only one confirmed protein shares a region of sequence identity of six amino acids. That protein, Zinc Finger Protein 646, has a region with a sequence identical to amino acids 13-18 of SEQ ID NO:1. Since this region of Zinc Finger Protein 646 does not contain the serine phosphorylation site, it is unlikely that an antibody generated to a phosphorylated form of a peptide having all or part of the amino acid sequence of SEQ ID NO:1 would have significant affinity to phosphorylated Sox9. A second hypothetical containing a six amino acid sequence with serine 211 was also identified. These data suggest that peptides having all or most of the amino acid sequence of SEQ ID NO:1 could be used to generate antibodies with high levels of specificity for Sox 9 phosphorylated at position 211. These data also suggest that peptides having a part of the amino acid sequence of SEQ ID NO:1 could be used to generate antibodies with high levels of specificity for Sox 9 unphosphorylated at position 211. In particular, these data suggest that all or part of amino acids 13-18 may not be suitable as an antigens for the generation of antibodies with high specificity to unphosphorylated Sox9.

The example employing Sox9 is, of course, only representative of methods that may be used to generate and/or identify antibodies with high levels of affinity and/or specificity for one or more antigens. Simlar methods may be used to obtain antibodies directed to other post-translational modifications of proteins. Thus, the invention includes compositions and methods for screening methods for generating and/or characterizing anibbody affinity and specificity to molecules that have related and/or similar conformations.

One example of another post-translational modification is methylation. A number of proteins are known to be subject to methylation. Protein post-translation modification has been found to be involved in the regulation of processes such as DNA repair and homologous recombination and arginine methylation is believed to be involved in this. Further, the arginine methyltransferase PRMT5 is believed to be a regulator of homologous recombination (HR)-mediated double-strand break (DSB) repair, mediated through its ability to methylate RUVBL1, a cofactor of the TIP60 complex. Further, PRMT5 is believed to target RUVBL1, which is believed to lead to the acetyltransferase activity of TIP60, promoting histone H4K16 acetylationBs. Thus, a prcess “cascade” is believed to occur. The invention provides compositions and methods for the identification of enzyme substrates, as well as the characterization fo antibodies for use in such methods.

With respect to enzyme substrates, the invention includes methods for IP of enzymes, followed by characterization of subtsrates bound to such enzymes. In many instances, the amount of substrate present in the IP may be low due to the often short duration of the enzyme substrate interaction. However, low prevalance proteins may be identified, then produced or purified for enzyme/substrate assay.

### EXAMPLES

### Example 1. Selection of Cell Models and Targets for IP-MS Antibody Validation

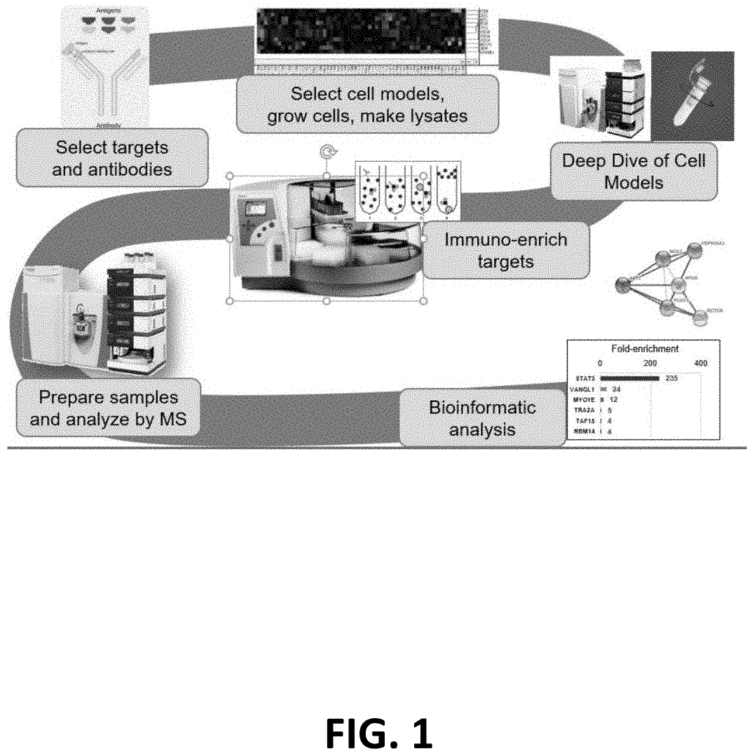

FIG. 1 presents an overview of the IP-MS antibody validation process. First, a protein target and antibodies were selected for validation. Pertinent cell models believed to contain the protein targets of interest were selected, grown, and lysates were prepared. A “deep-dive” analysis of a number of different cell lines was done to select pertinent cell lines for use with our initial IP-MS analyses. Antibodies thought to be specific to a certain target protein, for example via IP/Western, but not validated via IP-MS, were used to immuno-enrich the target protein from a cell lysate. Following IP, samples were analyzed by MS, and subjected to a novel bioinformatics analysis. Qualitative and quantitative data relating to the ability of each antibody to bind target protein was captured by this process, as was data relating non-target interacting proteins, as well as non-specific interacting protein partners. Background/non-specific binding was easily identified and discounted using this process.

The targets for various antibodies were prioritized based upon literature references, database mining, and consideration of signaling pathways and targeted genomic panels, such as the Thermo Scientific Ion Ampliseq panels for targeted gene amplification and next-generation DNA sequencing. The TP53 gene encoding p53 is the most highly referenced gene/protein in PubMed, with more than 7500 references, and therefore was identified for further study.

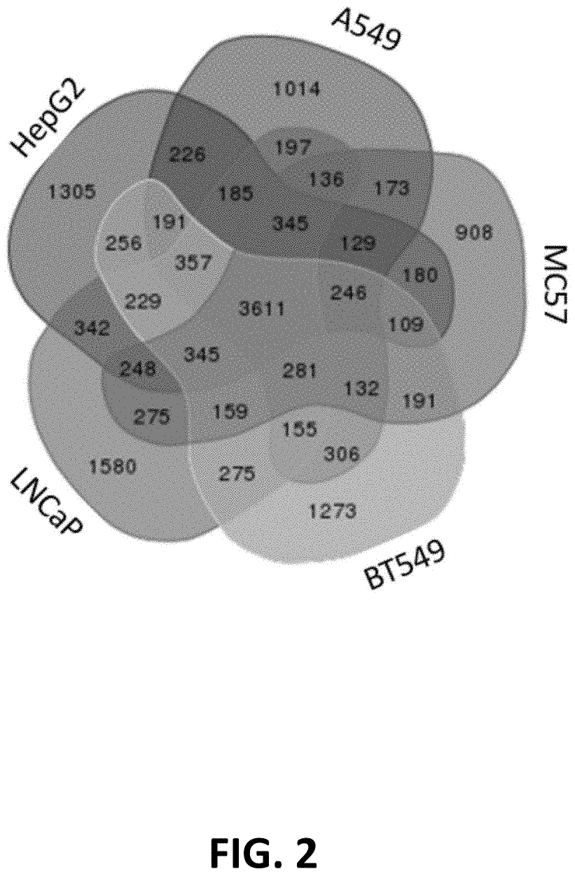

Once a list of target proteins was prioritized, literature resources and transcriptomic and proteomic databases were used to identify candidate complementary cell lines or biological samples likely to contain the most diverse and/or comprehensive set of protein targets. We initially selected twelve complementary cell lines likely to express more than 90% of the top 1000 most referenced genes or gene products for MS-based antigen capture, enrichment, and proteomic analysis: A549, BT549, HCT116, HEK293, HeLa, HepG2, Hs578T, LNCaP, MCF7, NIH3T3, SKMEL5, and SR. Data is presented for HepG2, A549, MCF7, BT549, and LNCaP cells in FIG. 2. Additional cell lines and biological samples are encompassed, and those named here are for exemplary purposes only.

The initially selected cell lines were grown as recommended to generate several hundred milligrams of each cellular protein lysate. Using state of the art sample preparation methods and instrumentation, each proteome was interrogated at two levels to identify the cell lines expressing each of these target proteins. Briefly, protein was solubilized, proteolytically digested with trypsin, and prepared for LC-MS analysis. Unfractionated and fractionated protein digests were analyzed directly by LC-MS. Fractionation improves the depth of proteome coverage by reducing the complexity of the protein lysate (peptides) using methods based on molecular weight, size, hydrophobicity, ion exchange binding, hydrophilic interactions, or affinity enrichment. Each unfractionated protein digest yielded about 3800-4500 unique protein family identifications, while each fractionated protein digest yielded about 7500-9000 unique protein families To assess the overall protein coverage and distribution, the identified proteins from each cell line were compared to determine pair-wise correlation scores between each of the different cell lines. These correlations ranged from 75-99%. When the overall protein identifications from the 5 least correlated cell lines were compared, 3611 proteins were observed in all five of these cell lines, with an additional 900-1500 unique proteins observed in each of these five diverse cell lines.

In addition to providing a comprehensive list of protein identifications from multiple cell lines, proteins were also quantified using a variety of label-free MS quantitation methods, including peptide signal intensity, label free protein quantitation (LFQ), and intensity-based absolute protein quantitation (iBAQ) values from MaxQuant. We found that LFQ and iBAQ provided useful complementary information, as the MaxQuant LFQ value relates to the relative molarity of a protein in a sample, while iBAQ considers the protein molecular weight and corresponds more closely with the relative mass of a protein, see Cox, J. and Mann M., Nat Biotech 26(12):1367-1372 (2008). The MS intensities of the top 3 peptides, the number of unique peptides identified, or the spectral count detected with Thermo Scientific Proteome Discoverer were also used, as these were a rapid and effective means of initial antibody screening.

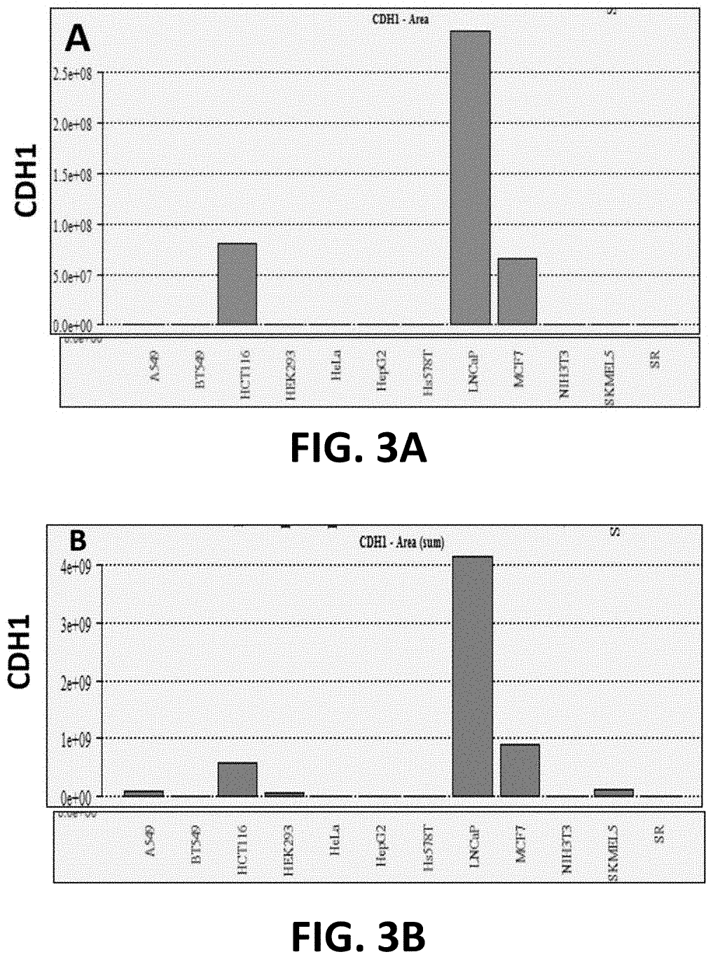

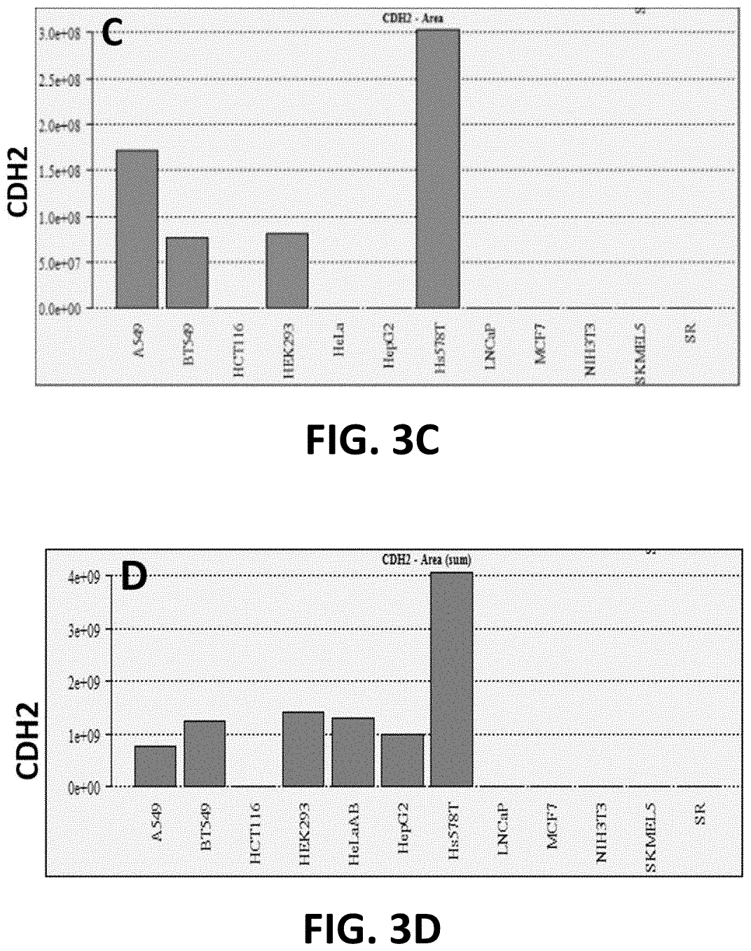

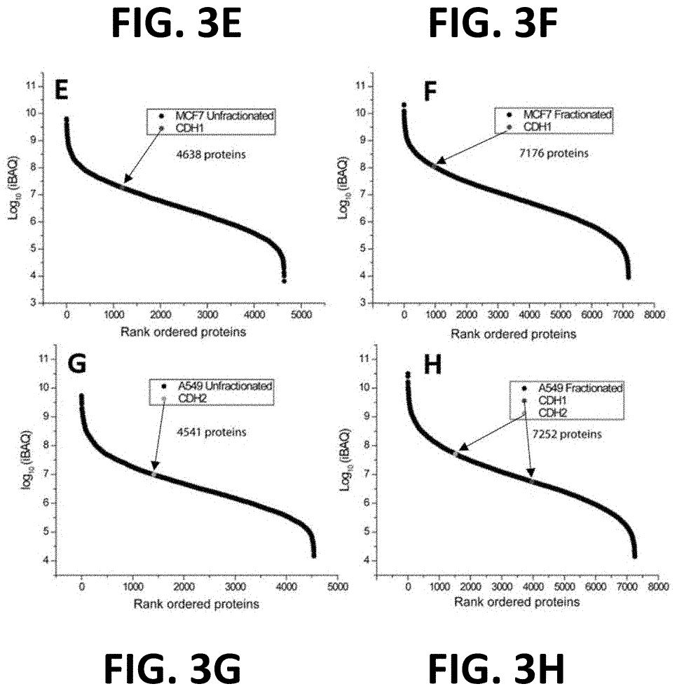

Using any of these label-free MS quantitation measures, target protein expression was ranked and compared across these cell lines, and appropriate cell lines for each target were selected for IP-MS antibody validation studies. For example, E-cadherin (CDH1) and N-cadherin (CDH2) had nearly opposite expression patterns between these 12 cells lines when the summed intensity of the three most intense peptides was quantified and plotted (FIGS. 3A-3D). E-cadherin (CDH1) was detected only in unfractionated HCT116, LNCaP, and MCF7 cells, while CDH2 was only seen in unfractionated A549, BT549, HEK293, and Hs578T cells. Both isoforms were detectable in several cell lines after fractionation and deeper MS analysis (FIGS. 3B and 3D), but neither cadherin isoform was detectable in NIH3T3 or SR cell lines. Furthermore, as cell lines with multiple gene copies and high over expression are inappropriate models for antibody validation, each native protein was ranked based upon MS signal intensity in order to select appropriate cells lines for antibody screening. For example, CDH1 was ranked about 1200 of about 4600 proteins in unfractionated MCF7 lysate (FIG. 3E) and about 800 of about 7100 proteins in the fractionated lysate (FIG. 3F). CDH2 was ranked about 1400 of about 4500 proteins in unfractionated A549 lysate (FIG. 3G) and about 1200 of about 7200 proteins in the fractionated lysate (FIG. 3H), while CDH1 was only detectable in A549 lysate after fractionation (rank of about 4000 of about 7200 proteins, FIG. 3H). This expression information was invaluable for the selection of cell models and validation of isoform-specific and pan-specific antibody selectivity. As a result, one or more cell lines for each target protein were chosen for validation based upon MS identification and levels of target protein expression.

Cell models and fractionation methods outlined in FIG. 1 are described below.

TABLE 1
Cell Models Used and Growth Conditions

| Cell Model | Tissue Type | Media | Product # | Insulin | Stimulation |
|---|---|---|---|---|---|
| HCT116 | Colon | McCoy's 5A | 16600-082 | N/A | ±IGF |
| A549 | Lung | Hamm's F12K | 21127-022 | N/A | ±IGF |
| MCF7 | Breast | DMEM | 11995-040 | 10 μg/mL | N/A |
| HepG2 | Liver | MEM | 11095-072 | N/A | ±Insulin |
| LNCaP | Prostate | RPMI-1640 | 11875-085 | N/A | ±IGF |
| NIH3T3 | Fibroblast | DMEM | 11995-040 | N/A | N/A |
| BT-549 | Breast | RPMI-1640 | 11875-085 | 0.023 IU/mL | N/A |
| SK MEL5 | Skin | DMEM | 11995-040 | N/A | N/A |
| Hs 578T | Breast | DMEM | 11995-040 | 10 μg/mL | N/A |
| SR | Lymphoblast | RPMI-1640 | 11875-085 | N/A | N/A |
| HeLa | Cervical | DMEM | 11995-040 | N/A | N/A |
| HEK293 | Kidney | DMEM | 11995-040 | N/A | N/A |

All cell lines were purchased from ATCC and grown in condition noted in Table 1. All media and cell growth products were purchased from Thermo Fisher Scientific (including trypsin (PN: 25200-056) and HBSS (PN: 14175-079)), and all media was supplemented with 10% FBS (PN: 16000-036), 1× Penicillin-Streptomycin (PN: 15140-163), and insulin if needed (PN: 12585014). Cells were grown to ˜80% confluency and passage 12-18 before lysis with Thermo Fisher Scientific IP Lysis Buffer (PN: 87788) and 1:100 HALT Protease and Phosphatase Inhibitor Cocktail (PN: 78445). If cells underwent stimulation, cells were starved in 0.1% Thermo Fisher Scientific Charcoal Stripped FBS (PN: SH30068.01) for 24 hours before stimulation with 100 ng/ml of IGF (Cell Signaling Technology PN: 8917SF) or 100 nM insulin (Tocris PN: 87788) for 15 minutes and then lysed immediately. Protein concentration was determined by Pierce BCA Protein Assay Kit (PN: 23225) using a Thermo Fisher scientific Multiskan GO instrument for measurement, and aliquots were stored at −80° C. until use.

### Lysate Sample Prep for Unfractionated and Fractionated Proteome Analysis

200-800 μg of lysate was further processed for analysis by mass spectrometry using the Pierce Mass Spec Sample Prep Kit for Cultured Cells (PN: 84840) as stated in the instruction booklet with proper scale up of reagents. After the final drying step, samples were reconstituted in 0.1% TFA and cleaned of incompatible salts, detergents, and other reagents using Pierce High pH Reverse-Phase Peptide Fractionation Kit (PN: 84868) with a custom protocol involving column conditioning, 3 washes with 0.1% TFA, and 3 elution steps with 50% acetonitrile and 0.1% TFA. Samples were dried in a vacuum concentrator and reconstituted in 200 μL of 0.1% TFA. 5 μl of sample in 45 μL of water (1:5 dilution) was aliquoted and the Pierce Quantitative Fluorometric Peptide Assay (PN:23290) was performed to determine peptide concentration as described in the instruction booklet.

For fractionation, 100 μg of digested peptide sample was fractionated with the Pierce High pH Reverse-Phase Peptide Fractionation Kit (PN: 84868), following the instruction booklet with the exception of a custom fractionation profile, as noted in Table 2.

TABLE 2
Custom Fractionation Profile

| | | Acetonitrile | Triethylamine (0.1%) |
|---|---|---|---|
| Fraction | Acetonitrile % | (100%), μL | in water, μL |
| 1 | 5.0% | 50 | 950 |
| 2 | 6.25% | 62.5 | 937.5 |
| 3 | 7.5% | 75 | 925 |
| 4 | 8.75% | 87.5 | 912.5 |
| 5 | 10.0% | 100 | 900 |
| 6 | 15.0% | 150 | 850 |
| 7 | 20.0% | 200 | 800 |
| 8 | 50.0% | 500 | 500 |

Fractionated samples were dried in a vacuum concentrator and reconstituted in 20 μL of 2% acetonitrile and 0.1% formic acid. Peptide concentration was measured with the Pierce Quantitative Fluorometric Peptide Assay (PN:23290) using 8 μL of sample in 16 μL of water (1:3 dilution). Unfractionated and fractionated samples were transferred into an autosampler vial for LC-MS analysis.

### LC-MS Analysis of Unfractionated and Fractionated Lysate Samples

2 μg of unfractionated and fractionated samples were analyzed by nanoLC-MS/MS on a THERMO SCIENTIFIC™ DIONEX™ ULTIMATE™ 3000 RSLCnano System and THERMO SCIENTIFIC™ Q EXACTIVE™ HF Hybrid Quadrupole-Orbitrap Mass Spectrometer using a Thermo Scientific EASY-Spray column (50 cm×75 μm ID, PepMap C18, 2 μm particles, 100 Å pore size, PN: ES803). The column temperature was maintained at 40° C. using an Easy Spray Ion Source (Thermo Scientific, PN: ES081) interfaced online with the mass spectrometer. Mobile phase A (0.1% Formic acid in water, LC-MS grade) and Mobile phase B (0.1% Formic acid in Acetonitrile (ACN), LC-MS grade) were used to buffer the pH in the two running buffers. The total gradient was 210 minutes followed by a 30 minutes washout and re-equilibration. In detail, the flow rate started at 300 nL/min and 2% ACN with a linear increase to 20% ACN over 170 minutes followed by 40 minutes linear increase to 32% ACN. The washout followed with a flow rate set to 400 nL/min at 95% ACN for 4 minutes followed by 24 minutes re-equilibration to 2% ACN.

The Q Exactive HF instrument (Thermo Scientific, Bremen, Germany) was freshly cleaned and calibrated using Tune (version 2.5 build 2042) instrument control software. Spray voltage was set to 1.9 kV, S-lens RF level at 60, and heated capillary at 275° C. Full scan resolutions were set to 120,000 at m/z 200. Full scan target was 1×10⁶ with a maximum IT fill time of 60 ms. Mass range was set to 400-1600. Target value for fragment scans was set at 1×10⁵, and intensity threshold was kept at 5×10⁴. Isolation width was set at 2.0 Th. The normalized collision energy was set at 27. Peptide match was set to preferred, and isotope exclusion was utilized. All data was acquired in profile mode using positive polarity.

### Example 2. IP/MS Protocol

The IP/MS protocol outlined in FIG. 1 was used to generate data in a number of different cells lines using the following methodologies.

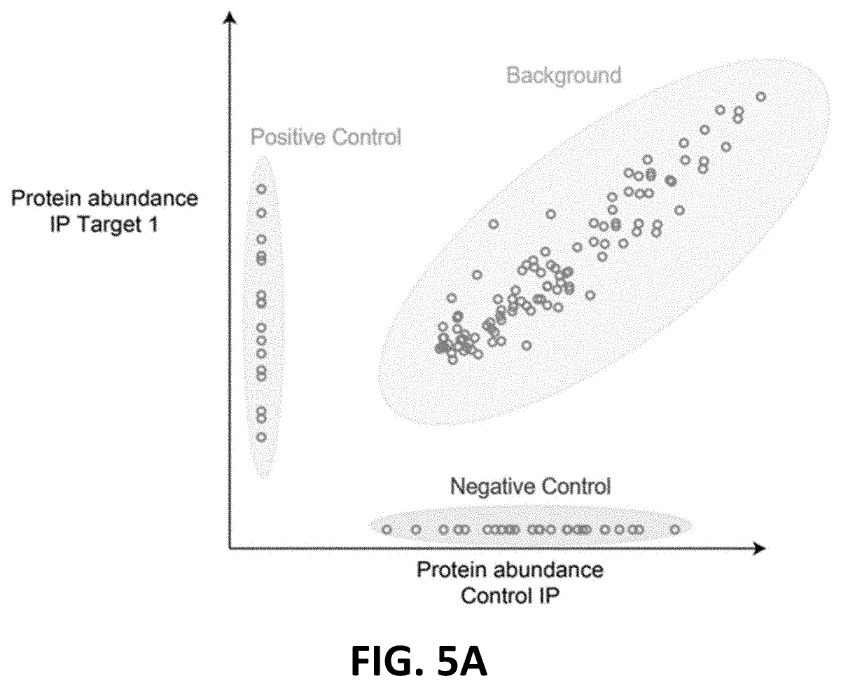

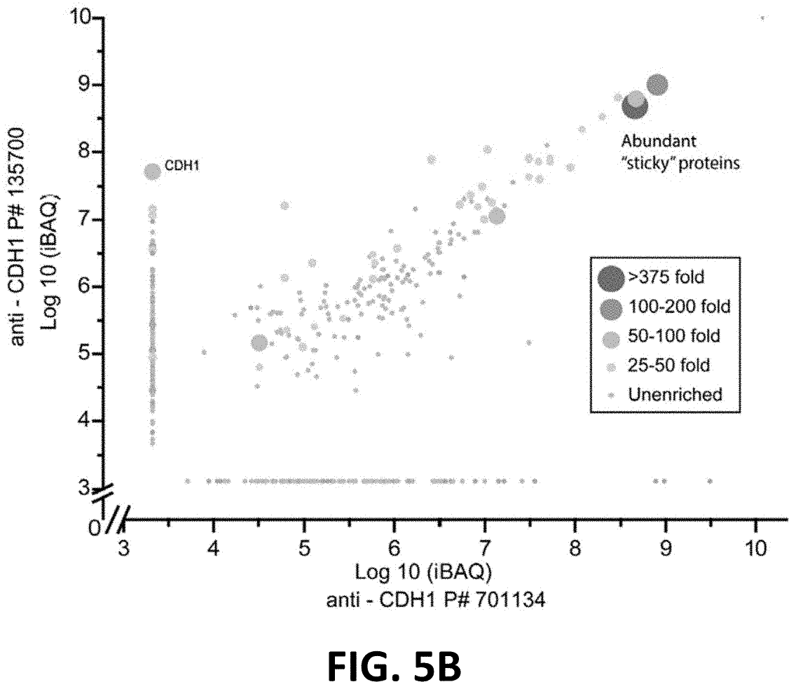

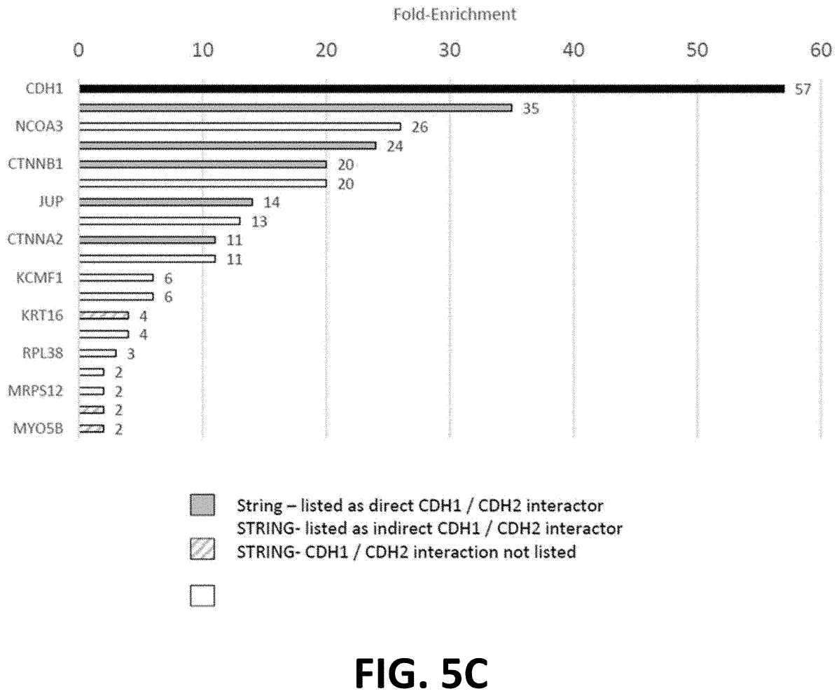

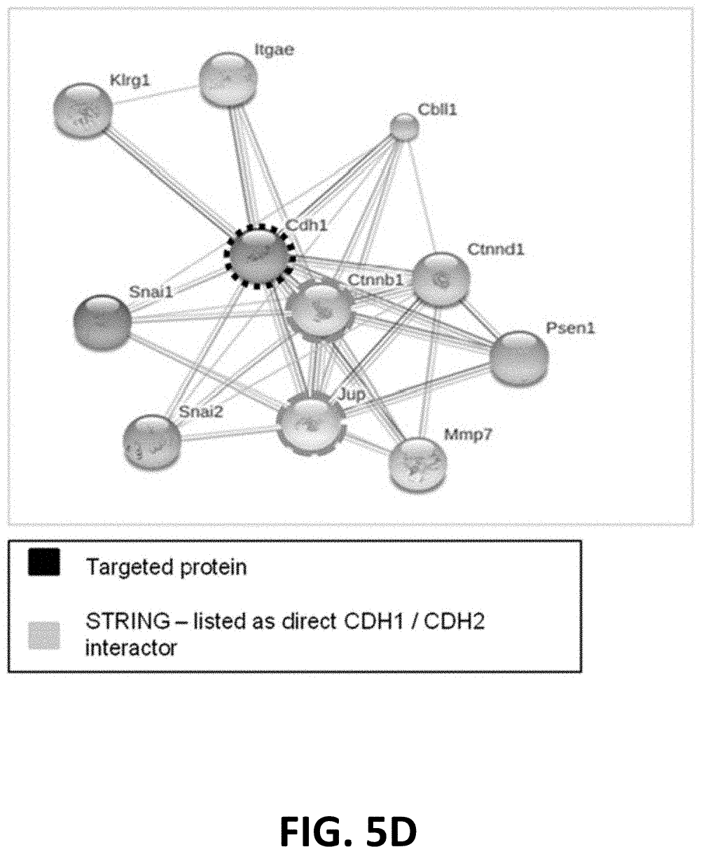

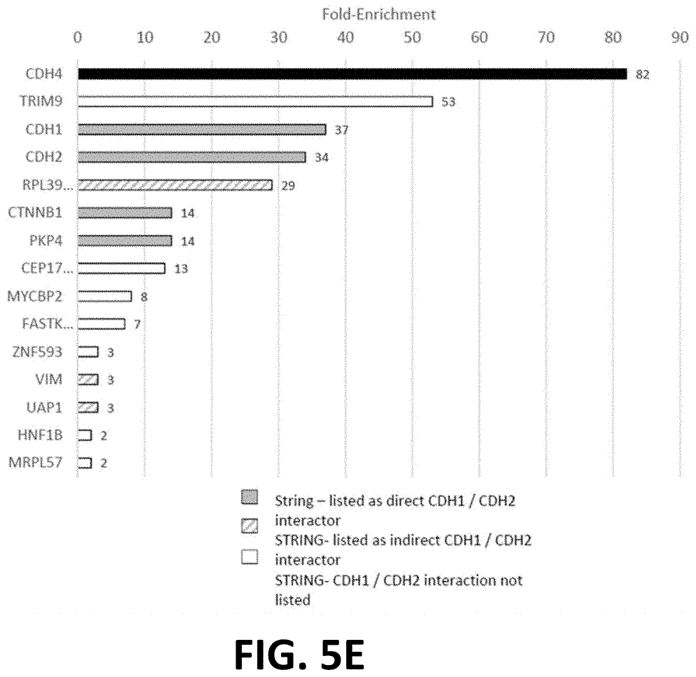

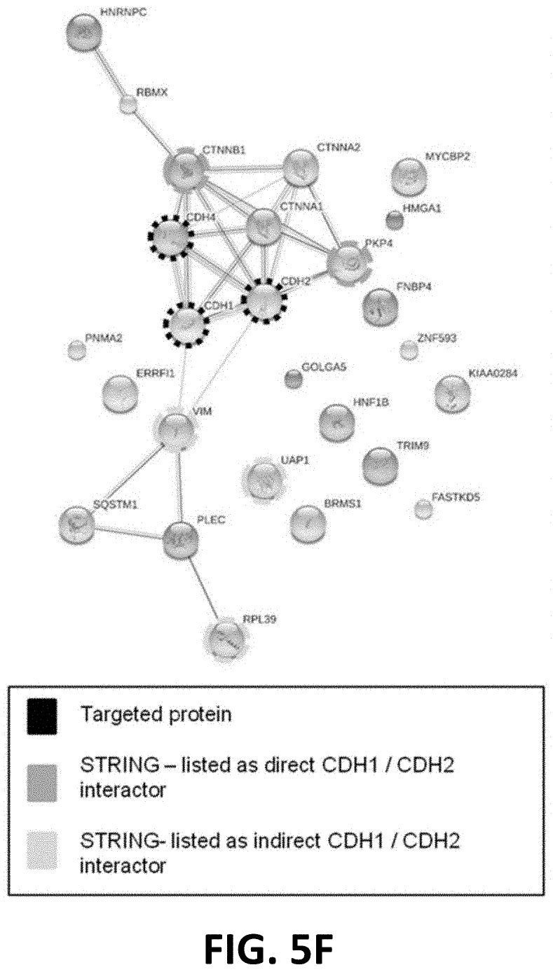

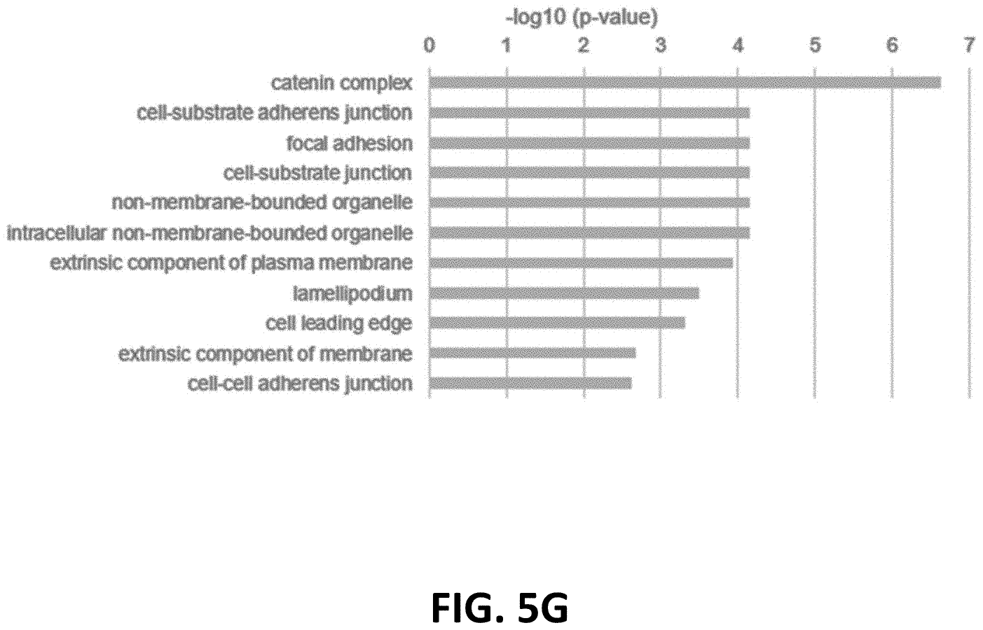

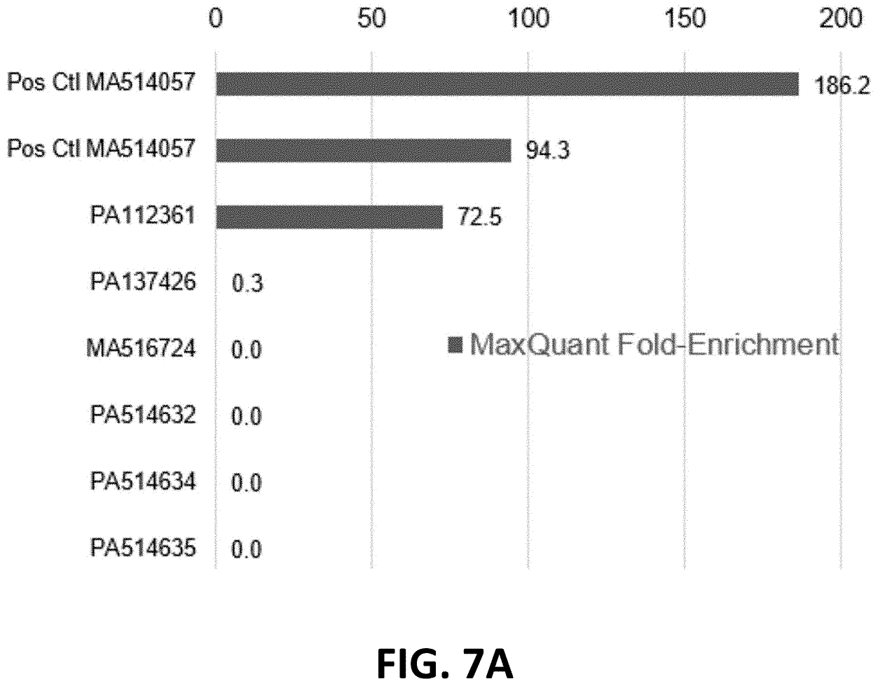

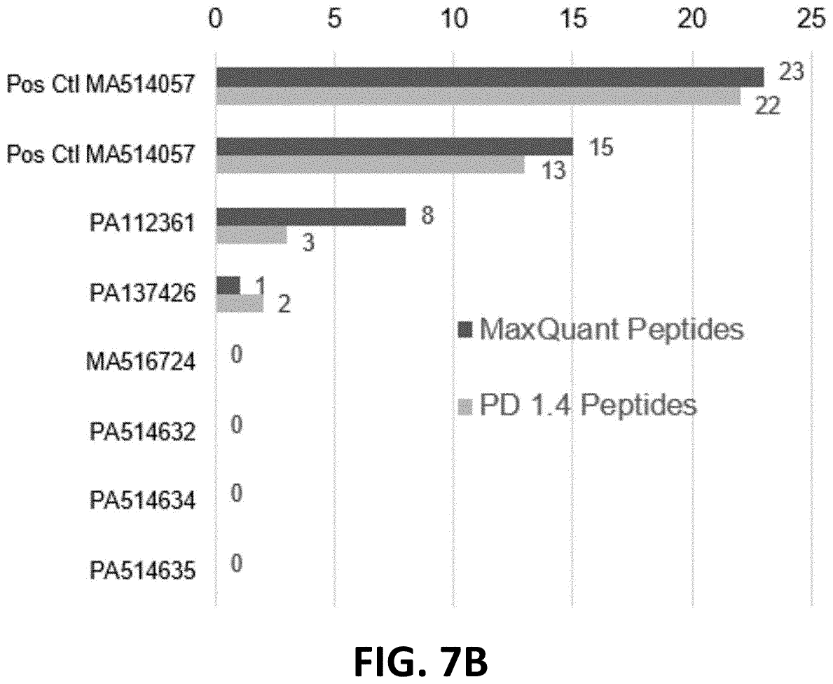

Antibodies against TP53, CDH1, CDH2 and CDKN1A were purchased from Thermo Fisher Scientific (Refer to FIGS. 5A, 5B, 7A). The Thermo Scientific Pierce MS-Compatible Magnetic IP Kit (Protein A/G) (Thermo Fisher Scientific, PN: 90409) was used to screen and verify antibodies as described in the instruction manual. 500 μg lysate and 5 μg of antibody were used for all experiments. IP eluates were dried in a vacuum concentrator and samples were processed by an in-solution digestion method as recommended in the instruction manual (Thermo Fisher Scientific, PN: 90409). Dried digested samples were resuspended in 13 μL of 4% acetonitrile and 0.2% formic acid and transferred into autosampler vials before LC-MS Analysis.

### LC-MS Analysis of IP-MS Samples

The IP-MS samples were analyzed by nanoLC-MS/MS using a THERMO SCIENTIFIC™ DIONEX™ ULTIMATE™ 3000 RSLCnano System coupled to THERMO™ SCIENTIFIC™ Q EXACTIVE™ HF Hybrid Quadrupole-Orbitrap Mass Spectrometer or THERMO™ SCIENTIFIC™ Q EXACTIVE™ Plus Orbitrap Mass Spectrometer. 7 μL of tryptic digest samples were desalted on-line using the Thermo Scientific Nano Trap Column (100 μm i.d.×2 cm, packed with ACCLAIM™ PEPMAP100™ C18, 5 μm, 100 Å, PN: 164564), and separated using a THERMO SCIENTIFIC™ EASY-SPRAY™ PEPMAP™ C18 column (15 cm×75 μm ID, 3 μm particles, 100 Å pore size, PN: ES803) with a total gradient time of 62 minutes. In detail, the flow rate started at 300 nL/min and 3% ACN with a linear increase to 25% ACN over 55 minutes followed by 7 minutes linear increase to 40% ACN. The column was washed with a flow rate set to 600 nL/min at 95% ACN for 3 minutes followed by 5 minutes re-equilibration to 3% ACN.

The Q Exactive HF and Q Exactive Plus instruments (Thermo Scientific, Bremen, Germany) were freshly cleaned and calibrated. Spray voltage was set to 1.9 kV, S-lens RF level at 60, and heated capillary at 275 ° C. Full scan resolutions were set to 70,000 at m/z 200 (Q Exactive Plus) and 60 000 at m/z 200 (Q Exactive HF). The full scan automatic gain control (AGC) target was set to 3×10⁶ with a maximum IT fill time of 50 ms for the Q Exactive Plus and 1×10⁶ with a maximum IT fill time of 60 ms for the Q Exactive HF. The mass ranges for both instruments were set to 400-1600 m/z. The target AGC value for fragment scans were set at 1×10⁵, and the intensity threshold were kept at 1×10⁴ (Q Exactive HF) and 3.3×10³ (Q Exactive Plus). Instrument isolation widths were set at 1.2 Th for Q Exactive HF and 2.0 Th for Q Exactive Plus. The normalized collision energy was set at 27 for both instruments. Peptide match was set to preferred, and isotope exclusion was utilized. All data was acquired in profile mode using positive polarity.

### Example 3. Target Identification by MS

The protein expression profiles from Example 1 and the techniques of Example 2 were used to assist in the validation of antibodies using an approach that combines immunoprecipitation with mass spectrometry (IP-MS). The key benefit of antibody verification by IP-MS is the identification of the native target protein and its isoforms and modifications. The MS results of this target identification can be assessed in several ways, including number of unique peptides, protein sequence coverage, number of spectra observed for peptides from the target protein (spectral count), or integrated MS signal intensities from a subset or all of the detected peptides, as described above. The relative performance of various antibodies for the same target can be easily compared regardless of the measurement approach. For example, immunoprecipitations with 13 IP/Western blot-validated antibodies to p53 protein (TP53) were assessed across days and across antibodies using the MS signal intensities for the three most intense p53 peptides (FIG. 5A). Results between days were highly reproducible, and 12 of these antibodies showed strong MS signals. This IP-MS antibody validation approach assesses antibody fit-for-purpose, provides definitive evidence of target protein capture, and readily permits antibody comparisons that may indicate relative antibody affinity.

While the IP-MS approach provides confidence in the antibody's protein target identification, the antibody, carrier proteins, and abundant non-specific proteins are also detected in the sample. These additional proteins may mask other protein antigens and can confuse the analysis. Importantly, an antibody must be shown to enrich its intended target, and ideally also co-enrich interaction partners relative to background proteins. To better quantify the performance and selectivity of an antibody and to normalize the results of antibodies against different targets across days, we utilized the concept of fold-enrichment. Calculations of fold-enrichment are commonly used to assess and optimize protein purification methods, and this approach can be used to assess an antibody's ability to enrich its native target from a biological matrix. Traditional antibody validation methods utilize comparisons of interacting proteins to databases, and have no mechanism to distinguish interacting proteins from non-specific binders. In contrast, fold-enrichment compares the abundance of a target protein captured by a test antibody to the abundance of the same target protein with a negative control IP or in a lysate sample with no IP. As such, non-specific binders are not enriched, or only slightly enriched, and can be validly overlooked in favor of enriched proteins. Traditional methods had no way to “subtract” these non-specific binders. The formula used for fold enrichment calculations is as follow:

$$F old enrichment = \frac{\frac{target protein abundance in the immunoprecipitate ( IP )}{total protein abundance in IP}}{\frac{target protein abundance in whole lysate}{total protein abundance in whole lysate}}$$

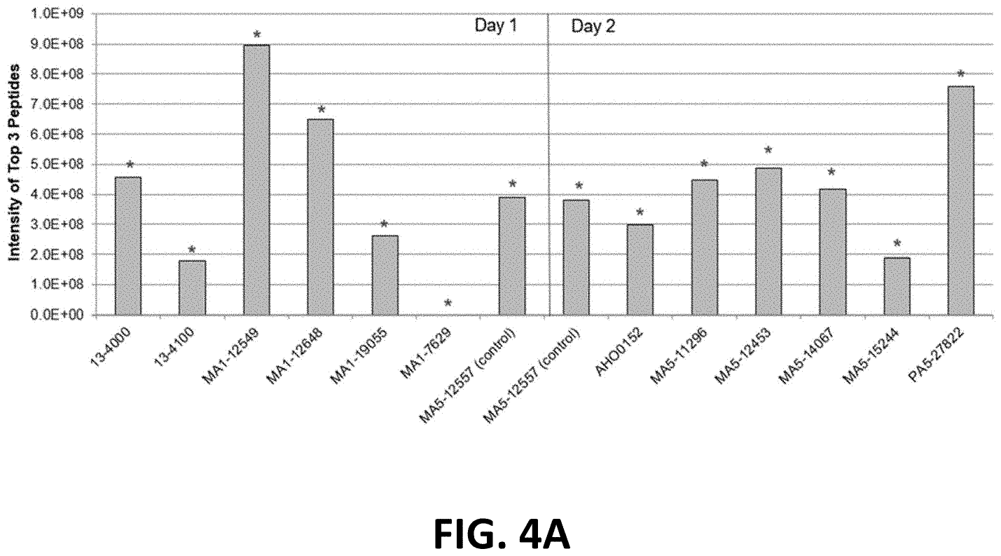

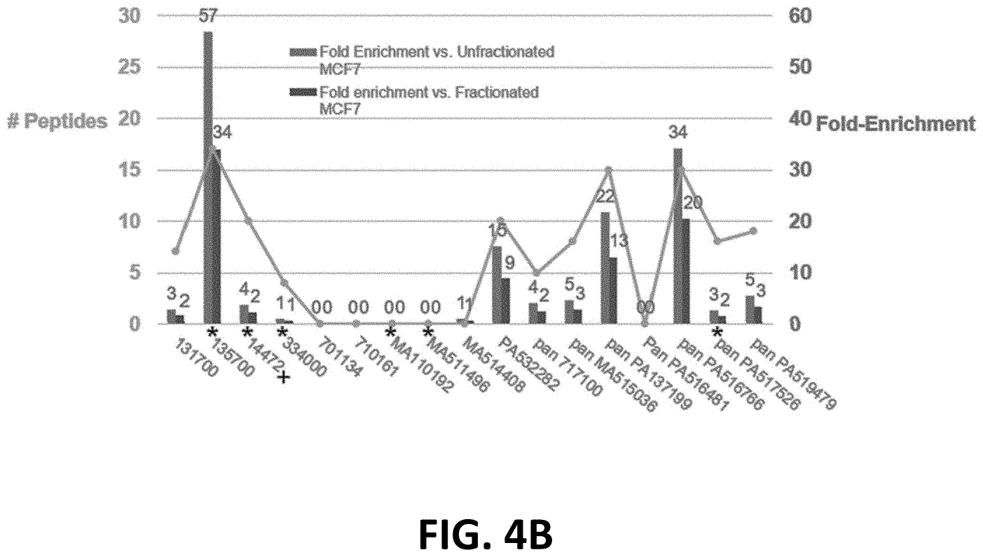

To assess the correlation between fold-enrichment and other measures of the protein quantitation, the fold-enrichment of CDH1 was calculated with 17 isoform-specific and pan-cadherin antibodies using LFQ values from unfractionated or fractionated lysates. The fold-enrichment values were compared with the number of detected CDH1 peptides, and a very high correlation was seen (FIG. 4B). The correlation thus validated the fold-enrichment method of validation. The calculated fold-enrichment was different depending on whether the MS results from the unfractionated or fractionated whole lysates were used. Therefore, if a protein was detected in the unfractionated lysate, that whole lysate MS signal was used for the fold-enrichment calculation. Otherwise the MS results from the deeper analysis of the fractionated lysate were used for the fold-enrichment calculation.

### Mass Spec Data Analysis and Visualization

MS data obtained from unfractionated lysate, fractionated lysates, and IP samples were analyzed using Proteome Discoverer 1.4 (release 1.14). A custom database of human, mouse, and rat proteomes (UniProt, assembled July 2014) was used for database search. Trypsin was selected as the enzyme used for digestion. During automated searching, concatenated target/decoy databases were generated to validate peptide-spectral matches (PSMs) and filter identifications to a 1% false discovery rate (FDR). MS spectra were searched using 20 ppm precursor mass tolerance and 0.03 Da fragment tolerance. The data was searched with a static modification of carbamidomethylation of cysteine residues, and dynamic modifications including the acetylation of protein N-termini, oxidation of methionine residues, and phosphorylation of serine, threonine, and tyrosine residues.

Protein groups of unfractionated, fractionated, and IP-MS sample data were exported and custom software was used to extract the unique peptide sequences, number of PSMs, and top 3 peptide peak areas for each identified protein. Top 3 peptide peak area was used to determine relative abundance of specific proteins across multiple cell lines.

Following PD 1.4 analysis, samples were searched using MaxQuant 1.5.3.51 to obtain relative quantification of peptides and proteins between unfractionated and fractionated proteome samples and compare these protein abundances to IP samples. The lysate used for one “set” of IP samples (where each antibody in the set was reported to recognize the same target protein) was identified and searched with the corresponding unfractionated and fractionated cell line data from the deep proteome analyses. Database searching was performed using an identical database and search parameters to the PD 1.4 searches. Label-free quantification (LFQ) was performed using a minimum LFQ ratio count of 2 and fast LFQ. Spectra were searched using a 20 ppm first search peptide tolerance and a 4.5 ppm main search peptide tolerance. MS/MS spectra were analyzed with a 20 ppm fragment match tolerance. Protein quantification was defined using a minimum threshold of 2 ratios, using unique and razor peptides for quantification. Large LFQ values were stabilized and required MS/MS for LFQ comparisons. iBAQ values were generated for all data and compared to raw protein intensities and LFQ values.

The MaxQuant data was manually analyzed to compare the intensities, LFQ, and iBAQ values obtained across the unfractionated, fractionated, and IP-MS samples. For each MS run searched, the LFQ abundance of each protein was extracted and divided by the summed abundance of all proteins identified to obtain a “fraction” of that protein's relative abundance versus every other protein identified in the sample. The relative fraction of the protein's abundance in an IP sample was then compared to the fraction of the protein in the deep proteome samples to observe whether this fraction increased, decreased, or stayed the same relative to the other proteins that were identified in each IP. In this way, a fold-enrichment was calculated for every protein in the IP samples, and this calculation was used to characterize the enrichment of putative antibody targets and known target-protein interactors. These fold-enrichment calculations were performed using both protein LFQ and iBAQ. Protein LFQ and iBAQ values were also used to generate scatterplots to characterize the specificity of antibodies used in IP. LFQ and iBAQ values were plotted to compare the relative abundances of proteins identified in a “test” IP (plotted on the y-axis) to those proteins identified in a negative control IP where the target was not identified (plotted on the x-axis). The negative control antibody was selected for chosen either because the antibody recognized a different target or did not identify the target that was pulled down by the test IP. Proteins observed uniquely in the test IP were ranked according to their fold-enrichment versus deep proteome samples. Fold-enrichment and scatterplot calculations were incorporated into a web application to streamline the generation of graphs for IP verification. The application was used to compare the fold-enrichment and scatterplots for antibodies using raw protein intensities, LFQ, and iBAQ values.

Proteins which were observed uniquely in test IPs and exhibited a >1-fold enrichment compared to deep proteome analysis were submitted to the STRING database (string-db.org) to probe known target-protein interactions. Protein interactions were selected against the Homo sapiens proteome. Proteins were plotted according to their known interactors using text mining, experimental verification, database annotation, co-expression, gene fusion, and co-occurrence data. Data was plotted with nodes representing proteins uniquely identified in the test IP and edges representing evidence of protein-protein interactions. Protein fold-enrichment bar charts were highlighted according to whether the identified protein was the putative antibody target or listed as a direct interactor with the target via the STRING database. Proteins were also highlighted to represent whether they were indirect interactors (i.e., listed as interacting with annotated target interactors) or were not listed as interacting via the STRING database. Network statistics from the STRING database were downloaded with enriched GO terms for cellular component, biological processes, molecular function, KEGG pathways, Pfam annotations, and InterPro classifications.

### Example 4. Background Subtraction with Scatter Plots

Protein immunoprecipitation with immobilized antibodies is a common method for targeted protein enrichment, but over one hundred background proteins are commonly identified by mass spectrometry even after stringent washing conditions. To better understand these background proteins and more easily identify specifically captured versus non-specifically captured proteins, MS intensity values were utilized to quantitatively compare the proteins immunoprecipitated with a specific antibody versus a negative control antibody (FIG. 5A). The resulting scatter plot of MS intensities showed three clusters: 1) specifically captured proteins that were only observed with the test antibody after immunoprecipitation (FIG. 5A, y-axis); 2) non-specifically captured proteins only observed with the negative control antibody immunoprecipitation (FIG. 5A, x-axis), and; 3) a scattering of proteins along the diagonal axis, which represented background proteins observed in both immunoprecipitations (FIG. 5A, diagonal area). This approach could be easily inversed to compare unrelated antibodies, or the results from many negative control antibodies could be used to remove common, non-specifically bound proteins. This quantitative analysis and visual representation quickly filtered the list of captured proteins identified with each antibody.

Scatter plot analysis was performed for CDH1 as shown in FIG. 5B. In this experiment, the 135700 antibody, which is believed to be specific for CDH1, was chosen as the “target IP” for at least the reason that it has been previously shown to induce the strong enrichment of CDH1 following IP among the anti-CDH1 antibodies tested (see FIG. 4B). The 701134 antibody was selected as the “control IP”, as it did not show any enrichment of CDH1 in FIG. 4B, and thus does not appear to have strong selectivity or affinity for CDH1. While a non-specific “control” antibody was chosen for this experiment, it should be noted that an antibody binding to the same target protein could be used as a “control” antibody with successful results.

To provide additional insight, the scatter plot results were compared against the fold-enrichment calculations for these same test and control anti-CDH1 antibodies (FIG. 5B, right side of graph). The results showed that CDH1 is highly enriched via IP-MS with the 135700 antibody, but not via IP-MS with the 701134 antibody (see CDH1 “dot” around 7.8 on the y-axis, and the absence of CDH1 “dot” along the x-axis). Interestingly, some of the most common background proteins (i.e., those falling along the diagonal) were significantly enriched when analyzed by the fold-enrichment method described above (see “dots” noted as abundant, “sticky” proteins in FIG. 5B). Therefore, a combination of fold enrichment and scatter plot analysis is particularly helpful in discriminating true protein targets from abundant non-specific binders. The presence of abundant non-specific proteins may be caused by specific binding to the magnetic bead resin or antibody isotype, and may depend on the cell type used in the sample preparation. In general, the scatter plot approach typically eliminated more than 90% of the identified proteins as non-specific binders (additional data not shown). Many, but not all, of the proteins falling along the diagonal in any particular scatter plot may be found in databases of background proteins such as, for example, the CRAPome database (see Mellacheruvu, D., et al., Nat Meth 10(8):730-736 (2013)). The use of the scatter plot provided a visual assessment of the repertoire of specific and background proteins, and its use in combination with fold enrichment data is particularly helpful in eliminating non-specific background proteins. Alternative quantitative tools to analyze protein affinity capture results, such as COMPASS, SAINT, and Perseus offer sophisticated scoring methods and analysis, but the results are still overwhelming and difficult for a non-expert to interpret. Exemplary alternative quantitative methods have been described in Marcon E, et al., Nature Methods 12:725-731 (2015); Keilhauer, E. C., et al., Mol Cell Proteomics 14(1):120-35 (2015); Teo, G., et al., Journal of Proteomics 100:37-43 (2014); and Sowa, M. E., et al., Cell 138(2):389-403 (2009). However, none of these tools utilize relative quantitative results between the native sample and the immune-enriched sample to determine fold-enrichment. This fold-enrichment calculation permits a direct measure of the absolute and relative specificity of antibodies for their targets.

The scatter plot analysis with fold-enrichment indicates that IP/MS is a powerful tool to verify that an antibody shows selectivity for the target protein over proteins that non-selectively associate with a negative control antibody.

### Example 5. Fold-Enrichment to Assess Selectivity and Identify Interaction Partners

Cell lines were chosen for antibody verification based upon a deep, MS-based proteome analysis, and more than 10,000 protein families were identified and quantified in whole cell lysates. Using these data, antibody performance was assessed quantitatively by calculating the fold-enrichment of all proteins identified in an immunoprecipitated sample. In this manner, the performance of different antibodies to the same target could be compared, and off-targets could be identified. For example, proteins immunoprecipitated from MCF7 cells with an IP/Western-validated CDH1 antibody (antibody 135700) were compared to proteins immunoprecipitated with another CDH1 antibody that was not validated for IP (antibody 701134). See, FIG. 5B. CDH1 was only identified with the IP-validated antibody, 135700, and fold-enrichment calculations identified a small subset of proteins that were also specifically enriched with this anti-CDH1 antibody, including alpha1-, alpha2- and beta1-catenin (CTNNA1, CTNNA2, CTNNB1) and plakoglobin (JUP, also known as gamma-catenin), as shown in FIG. 5C. These enriched proteins are known CDH1 interaction partners documented in BioGRID (http://thebiogrid.org/) and STRING (http://string-db.org/) protein interaction databases (with data from STRING shown in FIG. 5D). These proteins were not enriched by the control 701134 antibody.

In another experiment, proteins immunoprecipitated from A549 cells with a pan-specific anti-cadherin antibody (PA1-37199) were compared to proteins immunoprecipitated with another supposed pan-specific anti-cadherin antibody antibody (PA5-16481). FIG. 4B shows fold enrichment compared to number of peptides for each of these antibodies. PA1-37199 showed fold enrichment for CDH1, while PA5-16481 did not (see FIG. 4B). As shown in FIG. 5E, the PA1-37199 antibody enriched R-cadherin (CDH4), E-cadherin (CDH1), and N-Cadherin (CDH2) by 30- to 80-fold, as well as the TRIM9 protein, suggesting potential cross-reactivity or a novel interaction with TRIM9. A previous bioinformatic analysis of TRIM9 and its related proteins highlighted regions of structural similarity to the cadherin superfamily of proteins, potentially explaining the capture of TRIM9 protein with this pan-specific anticadherin antibody (see Short, K. M. and Cox T. C., J Biol Chem 281(13):8970-80 (2006)). Further bioinformatic analysis of the specifically immunoprecipitated and enriched proteins identified a large number of known protein interaction partners related to the catenin complex and cell adhesion (FIGS. 5F-5G). This data suggests that IP-MS is a highly specific and accurate method for validating an antibodies IP specificity, as well as accurately detecting meaningful protein-protein interactions.

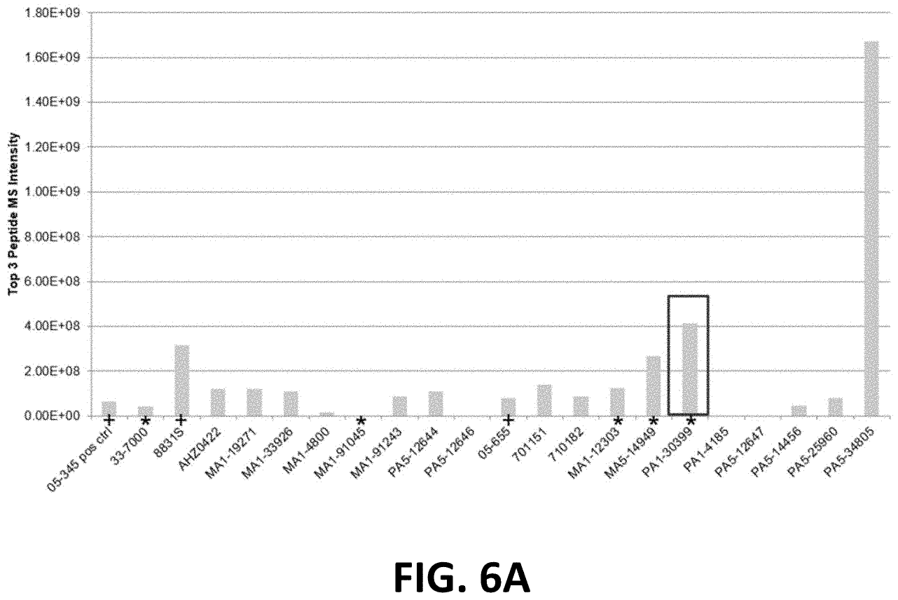

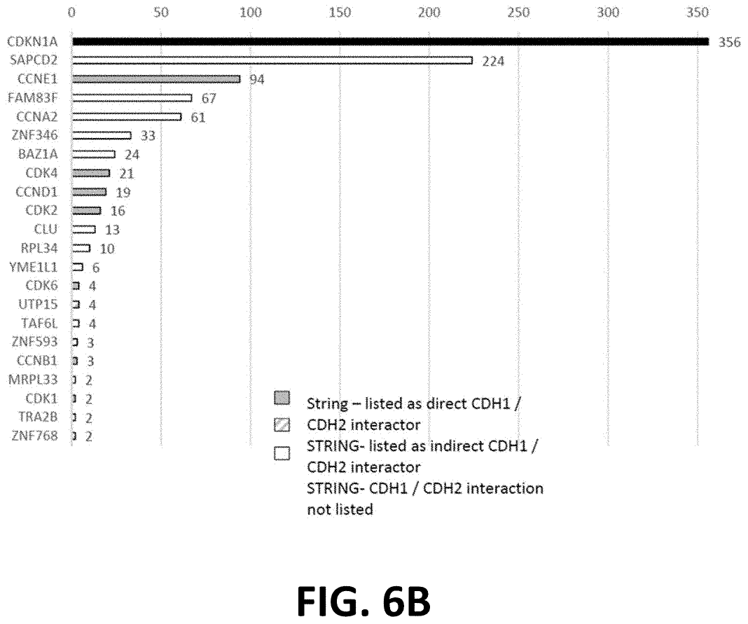

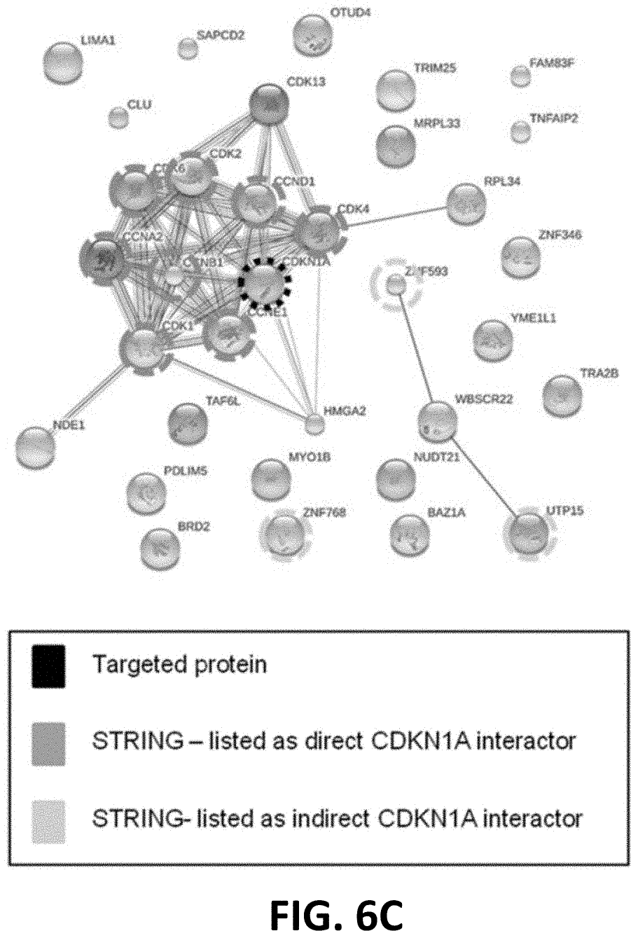

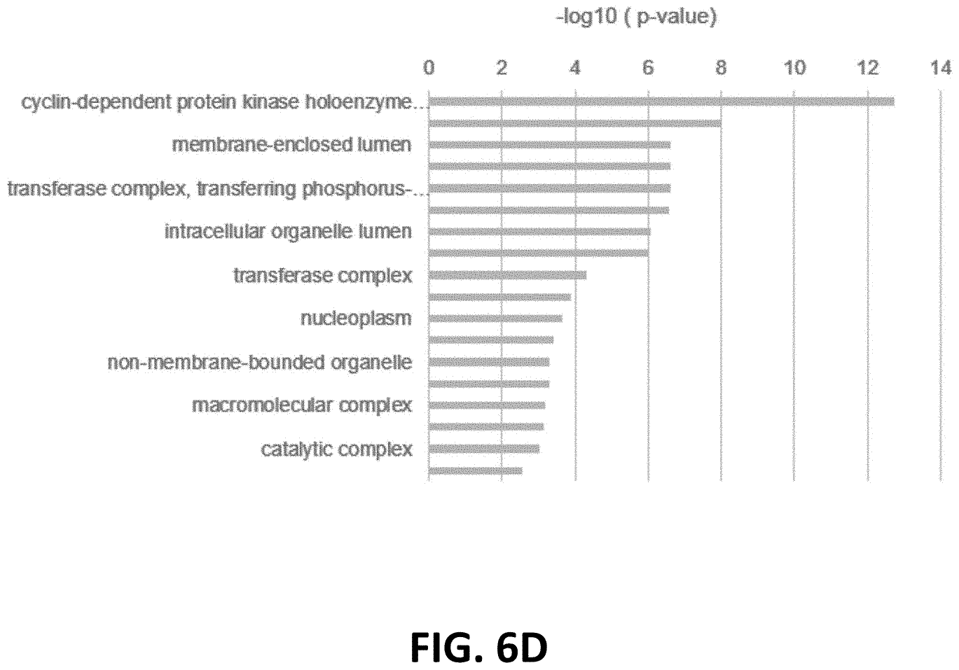

Next, 19 antibodies to p21Cip1, also known as cyclin-dependent kinase inhibitor 1 (CDKN1A), were compared against three IP-validated positive control antibodies using IP-MS (FIG. 7A). Four of the five previously IP-validated antibodies successfully captured the target, and an additional 11 antibodies not previously validated for IP also captured the target. As an example (shown in boxing in FIG. 6A), polyclonal antibody PA1-30399 enriched CDKN1A from HCT116 cells over 300-fold, along with many known protein interaction partners (FIG. 6B). For example, cyclin dependent kinases 1, 2, 4, and 6 (CDK1, CDK2, CDK4, CDK6) and cyclins A2, B1, D1, and E1 (CCNA2, CCNB1, CCND1, CCNE1) are all involved in regulating the cell cycle, and CDKN1A is a zinc finger-containing DNA binding protein that regulates the cell cycle by interacting with CDK4 for inhibit its phosphorylation of cyclin D. Interestingly, SAPCD2 is a tumor suppressor APC domain-containing protein that has never found been to interact with these other proteins. This protein is highly expressed in gastric cancer (see Xu et al., Oncogene 26, 7371-7379 (2007)), and the HCT116 cell line used for this antibody validation is derived from a colon cancer. This potential interaction could be tested by testing for co-capture of CDKN1A with an anti-SAPCD2 antibody. This battery of CDKN1A antibodies was also assessed for co-capture of SAPCD2 and other proteins, and its presence in many of the IP samples suggest that is is either common off-target or a strong interaction partner for CDKN1A. Three potential off-targets specific to this antibody include FAM83F, a poorly annotated protein that is phosphorlyated and acetylated, and ZNF346 and BAZ1A, which are both zinc finger proteins. These last two zinc finger proteins suggest that the epitope for this antibody may include the zinc finger domain of CDKN1A. Further comparison of enriched proteins with each antibody suggest that different paterns of protein interactors and epitopes may be detectable, potentially suggesting the ability to map antibodies to distinct epitopes and to identify complementary antibody pairs for “sandwich-type” antibody capture and detection applications. Bioinformatic analysis of the specifically captured and enriched proteins revealed many components of the cyclin-dependent protein kinase holoenzyme complex (FIGS. 6C-6D).

Next, a variety of anti-ERBB2 antibodies were evaluated for their ability to enrich for ERBB2 peptides following IP-MS.

FIG. 7A shows IP-MS results with a variety of anti-ERBB2 antibodies for the enrichment of ERBB2 peptides versus unfractionated samples. The MA514057 antibody (Thermo Fisher) was selected as a positive control, and two separate experiments showed a that an IP with this antibody produced a 94.3- and 186.2-fold enrichment versus unfractionated samples using MaxQuant analysis. IP-MS with the PA1-12361 antibody (Thermo Fisher) also showed high enrichment with a 72.5-fold enrichment versus unfractionated samples. Other anti-ERBB2 antibodies (PA1-37426, MA5-16724, PA5-14632, PA5-14634, and PA5-14635; all Thermo Fisher) did not show significant enrichment with IP-MS. Thus, the IP-MS process coupled with analysis using MaxQuant verified the ability of MA5-14057 and PA1-12361 to specifically interact with ERBB2, while indicating that other antibodies do not have substantial activity based on IP-MS analysis.

Shown below in Table 3 is a list of the anti-ErbB2 antibodies that were tested, along with the previously validated applications for each antibody. Applications include immunofluorescence (IF), immunocytochemistry (ICC), immunohistochemistry with frozen tissue or paraffin fixation (IHC, F or P), immunomicroscopy (IM), immunoprecipitation (IP), Western blotting (WB), enzyme-linked immunosorbent assay (ELISA), and fluorescence activated cell sorting (FACS).

TABLE 3
List of anti-ErbB2 antibodies, all of which are available from Thermo
Fisher Scientific, validated in the IP-MS methods described herein

| Lot No./ | | Validated |
|---|---|---|
| Cat. No. | Webname | Application |
| PG1875056/ | HER-2/ErbB2 | IF, ICC, IHC |
| MA5-12998 | Antibody | (F), IHC (P), IM |
| | (N12) | |
| 302P1507B/ | HER-2/ErbB2 | IF, ICC, IP |
| MA5-13003 | Antibody | |
| | (N24) | |
| RA2133893/ | HER-2/ErbB2 | IF, IHC (F), |
| MA5-12759 | Antibody | FACS, IP |
| | (9G6.10) | |
| 302X1502A/ | HER-2/ErbB2 | IF, IP |
| MA1-12691 | Antibody | |
| | (N24) | |
| 600P1508B/ | HER-2/ErbB2 | IP |
| MA5-13679 | Antibody | |
| | (L26) | |
| 307P1503D/ | HER-2/ErbB2 | IP |
| MA5-13032 | Antibody | |
| | (N28) | |
| 1350P1502F/ | HER-2/ErbB2 | WB |
| MA5-11976 | Antibody | |
| | (L87 + 2ERB19) | |
| QL2124771/ | HER-2/ErbB2 | WB |
| 700635 | Antibody | |
| | (40H87L57), | |
| | ABfinity ™ | |
| | Rabbit | |
| | Monoclonal | |
| QL225387/ | HER-2/ErbB2 | WB |
| 701399 | Antibody | |
| | (7H3L20), | |
| | ABfinity ™ | |
| | Rabbit | |
| | Monoclonal | |
| 1360705A/ | HER-2/ErbB2 | WB, ELISA |
| 710452 | Antibody | |
| | (7HCLC), | |
| | ABfinity ™ | |
| | Rabbit | |
| | Oligoclonal | |
| QJ2101963/ | HER-2/ErbB2 | WB, IF, ICC, IHC |
| MA5-15050 | Antibody | (F), IHC (P), FACS, IP |
| | (K.929.9) | |
| RA2131011/ | HER-2/ErbB2 | WB, IF, ICC, IHC |
| MA5-14057 | Antibody | (P), FACS, IP |
| | (e2-4001 + 3B5) | |
| QL2120086A/ | HER-2/ErbB2 | WB, IF, ICC, IHC |
| MA5-13105 | Antibody | (P), FACS, IP |
| | (e2-4001) | |
| QL2126071B/ | HER-2/ErbB2 | WB, IF, ICC, IHC, |
| MA5-13675 | Antibody | FACS, IP |
| | (3B5) | |
| 5153-1101/ | HER-2/ErbB2 | WB, IF, ICC, |
| PA5-20740 | Antibody | IHC, IP |
| 103P1510A/ | HER-2/ErbB2 | WB, IHC (P), IP |
| PA5-16305 | Antibody | |
| 0911RDU/ | HER-2/ErbB2 | WB, IHC (P, F), |
| MA1-82367 | Antibody | FACS, IP |
| | (ICR55) | |
| 325X1506B/ | HER-2/ErbB2 | WB, IHC, |
| AHO1011 | Antibody | FACS, IP |
| | (e2-4001) | |
| 9040P1506B/ | HER-2/ErbB2 | WB, IP |
| PA5-16774 | Antibody | |
| SH021028K/ | HER-2/ErbB2 | WB, IHC, FACS |
| PA5-14632 | Antibody | |
| SA120523CD/ | HER-2/ErbB2 | WB, IHC |
| PA5-14634 | Antibody | |
| SH080509D/ | HER-2/ErbB2 | WB, IHC, ICC, |
| PA5-14635 | Antibody | IF, FACS |
| 10378/ | HER-2/ErbB2 | WB |
| PA1-12361 | Antibody | |
| 812/MA5-16724 | HER-2/ErbB2 | IHC (F) |
| | Antibody | |
| | (ICR52) | |
| 150410LVE/ | HER-2/ErbB2 | IHC (P) |
| PA1-37426 | Antibody | |

In addition, FIG. 7B shows a number of ERBB2 peptides present in samples following IP-MS analysis. Again, IPs with the MA5-14057 and PA1-12361 antibodies led to the presence of ERBB2 peptides in IP samples. For all groups, the number of peptides present in the samples was greater using MaxQuant analysis versus PD1.4 analysis. We found that the searches with MaxQuant took longer than searches with PD1.4, but the MaxQuant searches identified more peptides for each protein and the LQF and iBAQ quantitation values were more reproducible. As a result, initial IP-MS results were typically search with PD1.4 first as a screening tool, then the LC-MS results from antibodies that appeared to work well were searched and quantified with MaxQuant to assess fold-enrichment and selectivity. The other anti-ERBB2 antibodies showed few or no ERBB2 peptides following IP-MS and analysis.

These data with ERBB2 confirm that the IP-MS verification process can rank multiple antibodies to this target by affinity and selectivity. For ERBB2, the antibodies with greatest affinity and selectivity were MA514057 and PA112361, while other antibodies did not demonstrate affinity and selectivity strong enough to induce enrichment of the target protein. Fold-enrichment based on IP-MS was confirmed to be a novel means of quantify antibody selectivity.

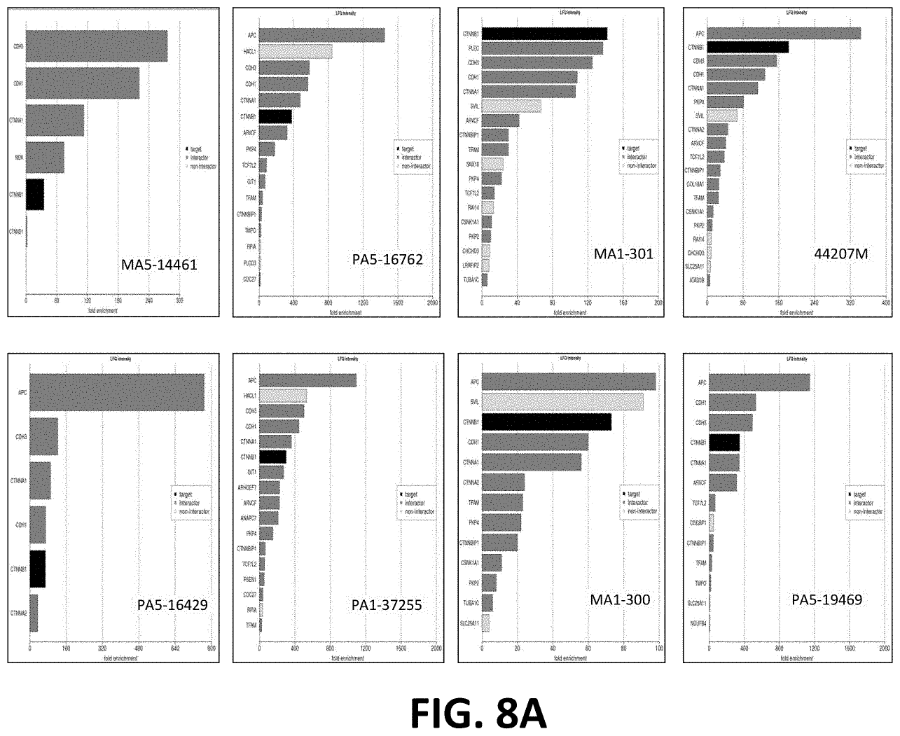

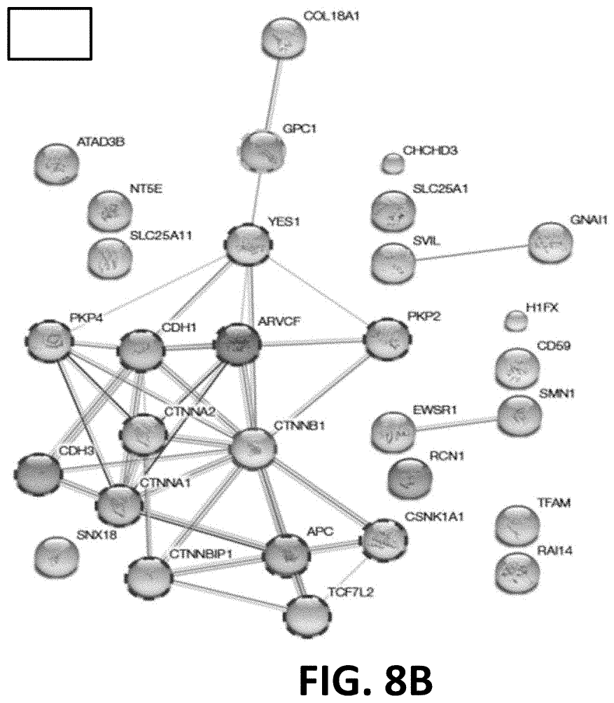

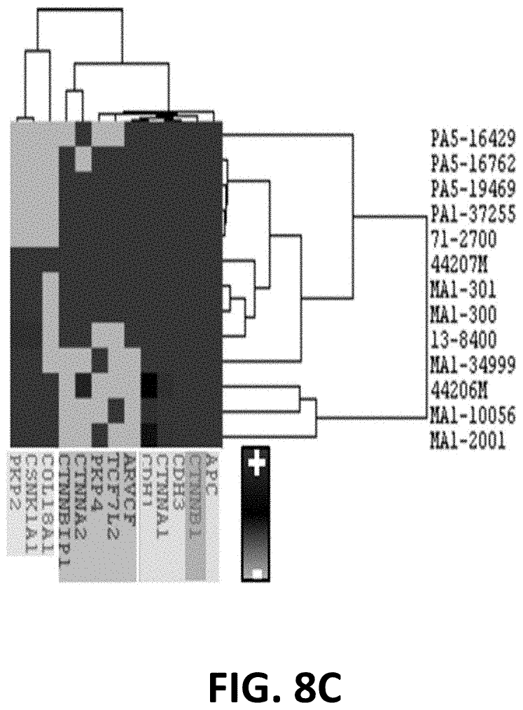

FIG. 8A shows the fold-enrichment of CTNNB1 from IP-MS experiments with eight antibodies to CTNNB1. The fold-enrichment of CTNNB1 varies from 30-fold to 370-fold depending on the antibody. A surface plasmon resonance (SPR) analysis of the binding properties of several of these antibodies to immobilized β-catenin protein suggests that the MS signal detected and resulting fold-enrichment is in part due to the range of dissociation rate constants observed (not shown). Higher dissociation rate constants correlated with lower MS signal. The immunoenriched complex on magnetic beads is washed multiple times prior to elution, which may result in this range of fold-enrichment values. Besides CTNNB1, APC, multiple cadherins (CDH1, CDH2, CDH4), and other known interactors are co-enriched with anti-CTNNB1 antibodies. FIG. 8B shows a STRING network diagram of known interactors of CTNNB1. These results complement the results shown in FIG. 5 that demonstrate the protein-protein interaction between CDH1 and CTNNB1 by the reverse experiment.

FIG. 8A also shows that independent antibodies to different epitope regions of the same target protein can enrich a common set of interactors but with unique fold-enrichment profiles. FIG. 8C shows the results of two-dimensional hierarchical clustering of the number of unique peptides detected for each interacting protein for each anti-CTNNB1 antibody. Cluster 3.0 (Laboratory of DNA Information Analysis, Human Genome Center, Institute of Medical Science, University of Tokyo) and Java TreeView (https://sourceforge.net/projects/jtreeview/) software were used to determine and draw the relationship between different interactors and different antibodies. This analysis suggests that different anti-CTNNB1 antibodies may recognize different sub-populations of the CTNNB1 interactome, perhaps due to recognition of unique CTNNB1 epitopes. The potential existence of distinct CTNNB1 interactome sub-populations may also contribute the to variable fold-enrichment of CTNNB1 observed, as they may indicate the relative abundance of these subpopulations in the lysate. Importantly, clustering approaches such as these described may be used to identify complementary antibodies that could be used together to provide greater specificity when used in combination in sandwich type antibody capture and detection assays, such as enzyme linked immunoassays (ELISAs) and bead-based immunoassays (e.g., Luminex assays).

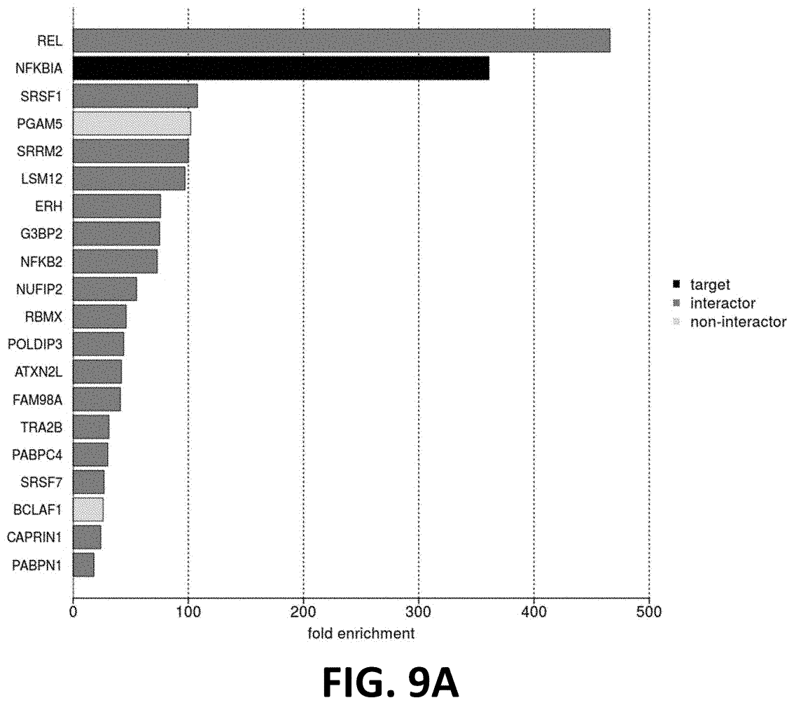

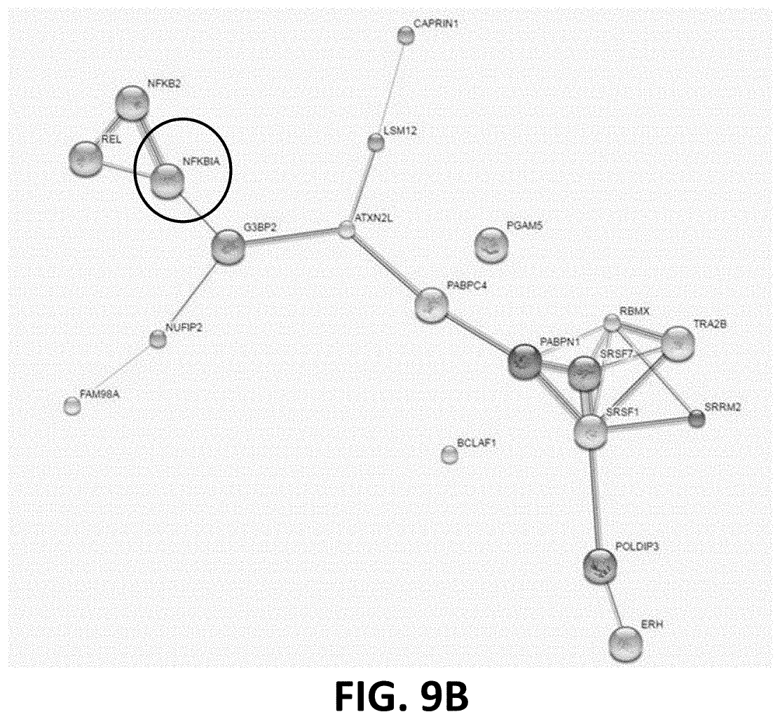

FIG. 9A shows the 360 fold-enrichment of NFKBIA with an antibody to the NFKBIA protein, as well as seventeen additional co-enriched proteins that are are predicted to interact with NFKBIA based upon the STRING network interaction diagram in FIG. 9B. These interactors play important roles in transcriptional regulation, RNA binding, RNA splicing, nuclear export of RNA, and translational regulation. Two possible explanations for the apparent enrichment of such a variety of interactors is that NFKBIA is in a >1 megadalton complex, or more likely, NFKBIA is bound to multiple RNA species at various stages of transcription, nuclear export, and translation, and that other proteins also binding to these RNA may be co-enriched.

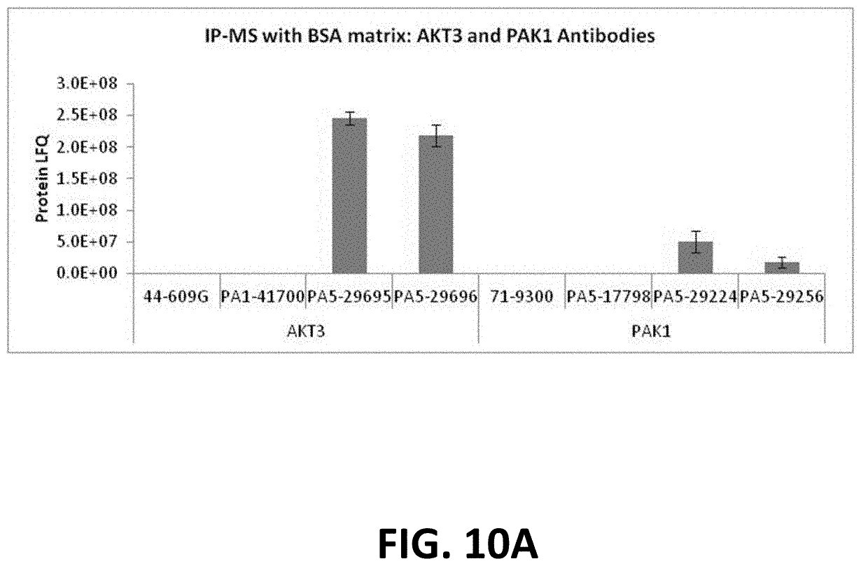

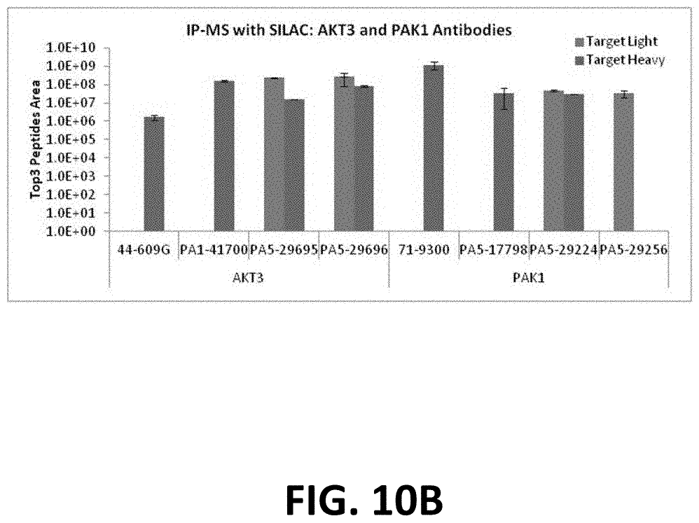

FIG. 10A shows four examples of affinity purified antibodies that appear to be contaminated with the peptide/protein affinity purification reagent. Four antibodies show detectable Akt3 or Pak1 targets in the IP-MS eluate despite the fact that no cell lysate was used in the experiment. The level of target protein determined by MaxQuant label free quantification (LFQ) in neat antibody preparations that were mixed with bovine serum albumin prior to IP-MS analysis demonstrate that significant amounts of the antibody target may be present in the antibody preparation. In cases where the antigen used for antibody development and purification was described, multiple peptides from the antigen sequence were present in the antibody preparation. To further verify this observation, FIG. 10B shows the intensity of light peptides enriched from the lysates of cells grown in media containing heavy isotope-labeled lysine and arginine amino acids. The light peptides appear to have been present in the antibody preparation used in the IP-MS reaction, but can be co-enriched with heavy native proteins in the cell lysate. The contamination may have come from leaching of the affinity material from the antibody purification resin during elution of the purified antibody. The contamination may may compete with the intended antigen and reduce the availability of antibody for antigen binding, it may increase the background signal in immunoassays, and it may interfere with targeted peptide detection in MS-based assays.

These data illustrate some of the benefits of antibody validation with IP-MS. IP-MS is a novel approach to antibody validation that uniquely verifies antibody capture performance, assesses antibody selectivity, identifies off-targets, and identifies interacting partners. This antibody validation approach is distinct from other protein-protein approaches because of the filtering and enrichment approaches used, as well as because the target proteins, off-targets, and interactors are identified in their native state (no N- or C-terminal tag) and are expressed at native levels with their interaction partners in a biologically relevant cell line or biological sample.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The following patent documents are incorporated herein by reference in their entireties: PCT/US2017/022062, filed Mar. 13, 2017; U.S. Provisional Application 62/308,051, filed Mar. 14, 2016; and U.S. Provisional Application 62/465,102, filed Feb. 28, 2017.

The invention is further represented by the following clauses:

Clause 1. A method for identifying proteins that specifically bind to an antibody comprising:

i) Selecting a test antibody;
ii) Preparing a cell lysate from a biological sample;
iii) Contacting the cell lysate with the test antibody, and immunoprecipitating the antibody and its protein binding partner(s);
iv) Analyzing the immunoprecipitated antibody and its protein binding partner(s) by mass spectrometry;
v) Determining the fold enrichment of the protein(s) bound to the test antibody as compared to the proteins in the cell lysate; and
vi) Identifying the proteins that specifically bind to the antibody, wherein proteins that specifically bind to the antibody are enriched as compared to proteins in the cell lysate.

Clause 2. A method for identifying proteins that specifically bind to an antibody comprising:

i) Selecting a test antibody;
ii) Preparing a first and second preparation of cell lysate from a biological sample, wherein the first and second preparations are nearly identical;
iii) Contacting the first cell lysate with the test antibody, and the second cell lysate with a second antibody, and immunoprecipitating the antibodies and their protein binding partner(s);
iv) Analyzing the immunoprecipitated test and second antibody and their protein binding partner(s) by mass spectrometry;
v) Plotting the intensity and/or fold enrichment of each protein identified by mass spectrometry as being bound to the test antibody on an x- or y-axis, and plotting the intensity or fold enrichment of each protein identified by mass spectrometry as being bound to the second antibody on the opposite axis; and
vi) Identifying the proteins that specifically bind to the test and second antibodies, wherein

a. proteins that specifically bind to the test and second antibody are those that do not display equal or nearly equal binding to both the test and second antibodies (those that fall along the diagonal when plotted);

b. proteins that specifically bind to the test antibody fall above the diagonal if plotted along the y-axis, or below the diagonal if plotted along the x-axis; and

c. proteins that specifically bind to the second antibody fall above the diagonal if plotted along the y-axis, or below the diagonal if plotted along the x-axis.

Clause 3. The method of clause 1 or clause 2, wherein the biological sample is a cell in cell culture, tissue, blood, serum, plasma, cerebral spinal fluid, urine, synovial fluid, peritoneal fluid, and other biofluids.

Clause 4. The method of clause 1 or clause 2, wherein biological sample can be stimulated or activated prior to contact with antibody.

Clause 5. The method of clause 4, wherein the stimulation is with a growth factor, hormone, toxin, or inhibitor.

Clause 6. The method of clause 3, wherein the cell in cell culture is a primary or secondary primary or immortal cell, or a stem cell.

Clause 7. The method of clause 6, wherein the cell is selected from A549, BT549, HCT116, HEK293, HeLa, HepG2, Hs578T, LNCaP, MCF7, NIH3T3, SKMEL5, and SR.

Clause 8. The method of clause 6, wherein the cell is selected from any cell in the NCI60 panel.

Clause 9. The method of clause 1 or clause 2, wherein the cell lysate is fractionated.

Clause 10. The method of clause 9, wherein fractioning comprises reducing the complexity of the cell lysate or digested cell lysate based on separation by molecular weight, size, hydrophobicity, ion exchange binding, hydrophilic interaction, or affinity enrichment.

Clause 11. The method of clause 1 or clause 2, wherein the fold enrichment is determined by the formula

$$fold enrichment = \frac{\frac{target protein abundance in the immunoprecipitate ( IP )}{total protein abundance in IP}}{\frac{target protein abundance in whole lysate}{total protein abundance in whole lysate}} ,$$

wherein a target protein is a protein bound to the test antibody.

Clause 12. The method of clause 1 or clause 2, wherein the protein(s) that specifically bind to the antibody are enriched about 5-fold or higher as compared to the protein(s) in the cell lysate.

Clause 13. The method of clause 12, wherein the fold enrichment is about 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200-fold higher as compared to the protein(s) in the cell lysate.

Clause 14. The method of clause 2, wherein the second antibody is:

a. an antibody that is believed to bind to a subset of the same protein or proteins as the test antibody; or

b. an antibody that is not believed to bind to the same protein or proteins as the test antibody.

Clause 15. The method of clause 14, wherein the second antibody is an isoform-specific antibody or a pan-specific antibody.

Clause 16. The method of clause 2, wherein plotting creates a scatter plot.

Clause 17. The method of clause 2, wherein the intensity is quantified.

Clause 18. The method of clause 11, wherein the intensity is quantified by label-free techniques or metabolic or chemical mass tagging techniques.

Clause 19. The method of clause 12, wherein the intensity is quantified by peptide signal intensity, label free protein quantitation (LFQ), intensity-based absolute protein quantitation (iBAQ), spectral counts, sequence coverage, number of unique peptides, or protein rank.

Clause 20. The method of clause 2, wherein the fold enrichment is plotted.

Clause 21. The method of clause 1 or clause 2, wherein the identified protein(s) are further characterized by sequencing.

Clause 22. The method of clause 1 or clause 2, wherein the identified protein(s) are post translationally modified.

Clause 23. The method of clause 1 or clause 2, wherein the antibody specifically binds to more than one target protein.

Clause 24. The method of clause 23, wherein the antibody is characterized according to its specificity to its target proteins, wherein a larger fold enrichment, or greater signal intensity, for one target protein as compared to another target protein means the antibody is more specific for that protein than for a protein with a smaller fold enrichment or lesser signal intensity.

Clause 25. The method of clause 23, wherein one target protein is the post translationally modified version of another target protein.

Clause 26. A method for determining the relative performance of more than one antibody comprising:

a. performing the methods of clause 1 for more than one test antibody, or

b. performing the methods of clause 2, and

c. comparing the performance of the test antibodies of clause 1, or the test and second antibody of clause 2, against each other, and ranking their performance based upon signal intensity, fold enrichment, sequence coverage, number of unique peptides, or spectral counts,

wherein one antibody performs better than another with respect to a particular target protein if its signal intensity, fold enrichment, sequence coverage, number of unique peptides, or spectral counts is greater than with another antibody.

Clause 27. The method of clause 2, wherein the test and second antibody are the same, and wherein protein in excess of what is needed to saturate the protein binding sites on the test antibody is added to the first cell lysate, but not the second cell lysate, prior to contact with the antibody, and wherein proteins that specifically bind to the test and second antibody are those that do not display equal or nearly equal binding to both the test and second antibodies (those that fall along the diagonal when plotted).

Clause 28. The method of any of the preceding clauses, wherein the mass spectrometry is selected from tandem mass spectrometry using data dependent acquisition and data independent acquisition.

Clause 29. The method of any of the preceding clauses, wherein the identified interaction partners, isoforms, or modifications may indicate distinguishable epitopes for different antibodies.

Clause 30. The method of any of the preceding clauses, wherein the immunoprecipitated antibody-target protein is digested prior to mass spectrometry.

Clause 31. The method of clause 30, wherein the digesting comprises a protease or chemical digest.

Clause 32. The method of clause 30, wherein the digestion is single or sequential.

Clause 33. The method of any of clauses 31 or 32, wherein the protease digestion is with trypsin, chymotrypsin, AspN, GluC, LysC, LysN, ArgC, proteinase K, pepsin, clostripain, elastase, GluC biocarb, LysC/P, LysN promisc, protein endopeptidase, staph protease or thermolysin.

Clause 34. The method of any of clauses 31 or 32, wherein the chemical cleavage is with CNBr, iodosobenzoate or formic acid.

Clause 35. The method of any of clauses 31 or 32, wherein the protease digest is a trypsin digest.

Clause 36. The method of any of the preceding clauses, further comprising desalting after immunoprecipitation or after digestion and prior to mass spectrometry.

Clause 37. A method for characterizing an antibody, the method comprising:

(a) determining the affinity of the antibody to an antigen, and

(b) determining the selectivity of the antibody for the antigen,

wherein the affinity of the antibody and/or the selectivity of the antibody are determined using immunoprecipitation-mass spectrometry (IP-MS), and

wherein the immunoprecipitate is generated by contacting the antigen with the antibody under conditions that allow for the formation on the immunoprecipitate between the antibody and the antigen.

Clause 38. The method of clause 37, wherein selectivity of the antibody for its binding partner is determined by the detection of binding to molecules in a cell lysate.

Clause 39. The method of clause 38, wherein the molecules are proteins.

Clause 40. The method of clause 38, wherein the cell lysate is derived from a cell of a species which expresses all of part of the antigen.

Clause 41. The method of clause 38, wherein selectivity is determined by western blot of the cell lysate.

Clause 42. The method of clause 38, wherein selectivity is determined using cells which expresses all of part of the antigen from more than one species.

Clause 43. The method of clause 42, wherein selectivity is determined by western blot of cell lysates from more than one species.

Clause 44. The method of clause 37, wherein selectivity is determined by generating an immunoprecipitate of a cell extract using the antigen, followed by identification and/or quantification of two or more non-antibody molecules present in the immunoprecipitate.

Clause 45. The method of clause 44, wherein the ratio of antigen/non-antibody molecules is calculated in the immunoprecipitate.

Clause 46. The method of clause 37, wherein the antibody is a high affinity antibody.

Clause 47. A method for preparing a matched set of antibodies, the method comprising:

(a) determining the affinity of each antibody for its respective antigen in a cell lysate,

(b) determining the selectivity of each antibody for its respective antigen in the cell lysate, and

(c) selecting antibodies to form the matched set,

wherein the affinity of the antibody and/or the selectivity of the antibody are determined using immunoprecipitation-mass spectrometry (IP-MS), and

wherein the matched set is composed of two or more antibodies that each have selectivity of at least 100 fold enrichment of for its respective antigen present in the cell lysate.

Clause 48. The method of clause 47, wherein the two or more antibodies that have affinities for their respective antigens with one log of each other.

Clause 49. The method of clause 47, wherein the matched set contains from three to ten antibodies.

Clause 50. The method of clause 47, wherein the antibodies bind to related targets.

Clause 51. The methods of clause 47, wherein the related targets are pre- and post-translationally modified forms of the same protein.

Clause 52. The methods of clause 51, wherein the pre-translationally modified form of the protein unphosphorylated and the post-translationally modified form of the protein phosphorylated.

Clause 53. A method for determining the selectivity of an antibody, the method comprising:

(a) contacting the antibody with a cell extract under conditions that allow for the formation on an immunoprecipitate between the antibody and one or more antigen in the cell extract,

(b) collecting the immunprecipitate formed in step (a), and

(c) indentifying one or more non-antibody molecules present in the immunprecipitate by mass spectrometry,

wherein the cell extract contains cell components from two or more cell types or one or more cell types from two or more species.

Clause 54. The method of clause 53, wherein the two or more cell types are from the same species.

Clause 55. The method of clause 53, wherein the cell types are obtained from two or more of the following tissues:

(a) muscular,

(b) connective,

(c) nervous, and

(d) epithelial.

Clause 56. The method of clause 53, wherein the connective tissue is blood.

Clause 57. The method of clause 53, wherein the two or more cell types are from different species.

Clause 58. The method of clause 57, wherein the two or more cell types from different species are obtained from two or more tissues from each species.

Clause 59. A method for determining the selectivity of an antibody, the method comprising:

(a) contacting the antibody with two or more proteins under conditions that allow for the formation on an immunoprecipitate between the antibody and one or more of the two or more proteins,

(b) collecting the immunprecipitate formed in step (a), and

(c) quantifying the amount of individual proteins present in the immunprecipitate by mass spectrometry.

Clause 60. A composition comprising:

(a) a cell extract obtained from one or more cell types, and

(b) an exogenously added antibody,

wherein the cell types are from two or more different species.

Clause 61. The composition of clause 60, wherein the cell extract is prepared from cell lysates.

Clause 62. The composition of clause 61, wherein the cell lysates are obtained by lysing cells of the two or more cells types, followed by centrifugation of the resulting lysate to remove insoluble matter.

Clause 63. The composition of clause 60, wherein the centrifugation is performed at greater or equal to 10,000×g for at least 15 minutes.

Clause 64. The composition of clause 60, wherein the antibody has affinity for at least one protein present in the cell extract.

Clause 65. A method for identifying an antibody that selectively binds to target molecules of cells obtained from different species, the method comprising:

(a) contacting the antibody with two or more cell lysates generated from cell of different species under conditions that allow for the formation on two or more immunoprecipitates between the antibody and one or more target molecule present in each cell lysate,

(b) collecting the immunoprecipitate from each cell lysate, and

(c) determining the fold purification for the target molecules in each immunoprecipitate by mass spectrometry.

Clause 66. The method of clause 65, wherein the species are selected from the group consisting of:

(a) Homo sapiens,

(b) Oryctolagus cuniculus,

(c) Mus musculus, and

(d) Rattus norvegicus.

Clause 67. The method of clause 65, wherein the antibody is generated in response to an epitope or a protein that is conserved across the different species from which the cell lysates are obtained.

Clause 68. The method of clause 67, wherein the epitope is from a protein or the protein is in a category selected from the group consisting of:

(a) heat shock proteins,

(b) polymerases,

(c) cell surface receptors,

(d) transcription factors,

(e) kinases,

(f) dephosphorylases,

(g) membrane associated transporters, and

(h) zinc finger proteins.

What is claimed is:

1. A method for identifying proteins that specifically bind to an antibody comprising:
i) Selecting a test antibody;
ii) Preparing a cell lysate from a biological sample;
iii) Contacting the cell lysate with the test antibody, and immunoprecipitating the antibody and its protein binding partner(s);
iv) Analyzing the immunoprecipitated antibody and its protein binding partner(s) by mass spectrometry;
v) Determining the fold enrichment of the protein(s) bound to the test antibody as compared to the proteins in the cell lysate; and
vi) Identifying the proteins that specifically bind to the antibody, wherein proteins that specifically bind to the antibody are enriched as compared to proteins in the cell lysate.

2. A method for identifying proteins that specifically bind to an antibody comprising:
i) Selecting a test antibody;
ii) Preparing a first and second preparation of cell lysate from a biological sample, wherein the first and second preparations are nearly identical;
iii) Contacting the first cell lysate with the test antibody, and the second cell lysate with a second antibody, and immunoprecipitating the antibodies and their protein binding partner(s);
iv) Analyzing the immunoprecipitated test and second antibody and their protein binding partner(s) by mass spectrometry;
v) Plotting the intensity and/or fold enrichment of each protein identified by mass spectrometry as being bound to the test antibody on an x- or y-axis, and plotting the intensity or fold enrichment of each protein identified by mass spectrometry as being bound to the second antibody on the opposite axis; and
vi) Identifying the proteins that specifically bind to the test and second antibodies, wherein
a. proteins that specifically bind to the test and second antibody are those that do not display equal or nearly equal binding to both the test and second antibodies (those that fall along the diagonal when plotted);
b. proteins that specifically bind to the test antibody fall above the diagonal if plotted along the y-axis, or below the diagonal if plotted along the x-axis; and
c. proteins that specifically bind to the second antibody fall above the diagonal if plotted along the y-axis, or below the diagonal if plotted along the x-axis.

3. The method of claim 1 and claim 2, wherein the biological sample is a cell in cell culture, tissue, blood, serum, plasma, cerebral spinal fluid, urine, synovial fluid, peritoneal fluid, and other biofluids.

4. The method of claim 1 and claim 2, wherein biological sample can be stimulated or activated prior to contact with antibody.

5. The method of claim 4, wherein the stimulation is with a growth factor, hormone, toxin, or inhibitor.

6. The method of claim 3, wherein the cell in cell culture is a primary or secondary primary or immortal cell, or a stem cell.

7. The method of claim 6, wherein the cell is selected from A549, BT549, HCT116, HEK293, HeLa, HepG2, Hs578T, LNCaP, MCF7, NIH3T3, SKMEL5, and SR.

8. The method of claim 6, wherein the cell is selected from any cell in the NCI60 panel.

9. The method of claim 1 and claim 2, wherein the cell lysate is fractionated.

10. The method of claim 9, wherein fractioning comprises reducing the complexity of the cell lysate or digested cell lysate based on separation by molecular weight, size, hydrophobicity, ion exchange binding, hydrophilic interaction, or affinity enrichment.

11. The method of claim 1 and claim 2, wherein the fold enrichment is determined by the formula
$$fold enrichment = \frac{\frac{target protein abundance in the immunoprecipitate ( IP )}{total protein abundance in IP}}{\frac{target protein abundance in whole lysate}{total protein abundance in whole lysate}} ,$$
wherein a target protein is a protein bound to the test antibody.

12. The method of claim 1 and claim 2, wherein the protein(s) that specifically bind to the antibody are enriched about 5-fold or higher as compared to the protein(s) in the cell lysate.

13. The method of claim 12, wherein the fold enrichment is about 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200-fold higher as compared to the protein(s) in the cell lysate.

14. The method of claim 2, wherein the second antibody is
a. an antibody that is believed to bind to a subset of the same protein or proteins as the test antibody; or
b. an antibody that is not believed to bind to the same protein or proteins as the test antibody.

15. The method of claim 14, wherein the second antibody is an isoform-specific antibody or a pan-specific antibody.

16. The method of claim 2, wherein plotting creates a scatter plot.

17. The method of claim 2, wherein the intensity is quantified.

18. The method of claim 11, wherein the intensity is quantified by label-free techniques or metabolic or chemical mass tagging techniques.

19. The method of claim 12, wherein the intensity is quantified by peptide signal intensity, label free protein quantitation (LFQ), intensity-based absolute protein quantitation (iBAQ), spectral counts, sequence coverage, number of unique peptides, or protein rank.

20. The method of claim 2, wherein the fold enrichment is plotted.

21. The method of claim 1 and claim 2, wherein the identified protein(s) are further characterized by sequencing.

22. The method of claim 1 and claim 2, wherein the identified protein(s) are post translationally modified.

23. The method of claim 1 and claim 2, wherein the antibody specifically binds to more than one target protein.

24. The method of claim 23, wherein the antibody is characterized according to its specificity to its target proteins, wherein a larger fold enrichment, or greater signal intensity, for one target protein as compared to another target protein means the antibody is more specific for that protein than for a protein with a smaller fold enrichment or lesser signal intensity.

25. The method of claim 23, wherein one target protein is the post translationally modified version of another target protein.

26. A method for determining the relative performance of more than one antibody comprising:
a. performing the methods of claim 1 for more than one test antibody, or
b. performing the methods of claim 2, and
comparing the performance of the test antibodies of claim 1, or the test and second antibody of claim 2, against each other, and ranking their performance based upon signal intensity, fold enrichment, sequence coverage, number of unique peptides, or spectral counts, wherein one antibody performs better than another with respect to a particular target protein if its signal intensity, fold enrichment, sequence coverage, number of unique peptides, or spectral counts is greater than with another antibody.

27. The method of claim 2, wherein the test and second antibody are the same, and wherein protein in excess of what is needed to saturate the protein binding sites on the test antibody is added to the first cell lysate, but not the second cell lysate, prior to contact with the antibody, and wherein proteins that specifically bind to the test and second antibody are those that do not display equal or nearly equal binding to both the test and second antibodies (those that fall along the diagonal when plotted).

28. The method of any of the preceding claims, wherein the mass spectrometry is selected from tandem mass spectrometry using data dependent acquisition and data independent acquisition.

29. The method of any of the preceding claims, wherein the identified interaction partners, isoforms, or modifications may indicate distinguishable epitopes for different antibodies.

30. The method of any of the preceding claims, wherein the immunoprecipitated antibody-target protein is digested prior to mass spectrometry.

31. The method of claim 30, wherein the digesting comprises a protease or chemical digest.

32. The method of claim 30, wherein the digestion is single or sequential.

33. The method of any of claims 31 and 32, wherein the protease digestion is with trypsin, chymotrypsin, AspN, GluC, LysC, LysN, ArgC, proteinase K, pepsin, clostripain, elastase, GluC biocarb, LysC/P, LysN promisc, protein endopeptidase, staph protease or thermolysin.

34. The method of any of claims 31 and 32, wherein the chemical cleavage is with CNBr, iodosobenzoate or formic acid.

35. The method of any of claims 31 and 32, wherein the protease digest is a trypsin digest.

36. The method of any of the preceding claims, further comprising desalting after immunoprecipitation or after digestion and prior to mass spectrometry.

37. A method for characterizing an antibody, the method comprising:
(a) determining the affinity of the antibody to an antigen, and
(b) determining the selectivity of the antibody for the antigen,
wherein the affinity of the antibody and/or the selectivity of the antibody are determined using immunoprecipitation-mass spectrometry (IP-MS), and
wherein the immunoprecipitate is generated by contacting the antigen with the antibody under conditions that allow for the formation on the immunoprecipitate between the antibody and the antigen.

38. The method of claim 37, wherein selectivity of the antibody for its binding partner is determined by the detection of binding to molecules in a cell lysate.

39. The method of claim 38, wherein the molecules are proteins.

40. The method of claim 38, wherein the cell lysate is derived from a cell of a species which expresses all of part of the antigen.

41. The method of claim 38, wherein selectivity is determined by western blot of the cell lysate.

42. The method of claim 38, wherein selectivity is determined using cells which expresses all of part of the antigen from more than one species.

43. The method of claim 42, wherein selectivity is determined by western blot of cell lysates from more than one species.

44. The method of claim 37, wherein selectivity is determined by generating an immunoprecipitate of a cell extract using the antigen, followed by identification and/or quantification of two or more non-antibody molecules present in the immunoprecipitate.

45. The method of claim 44, wherein the ratio of antigen/non-antibody molecules is calculated in the immunoprecipitate.

46. The method of claim 37, wherein the antibody is a high affinity antibody.

47. A method for preparing a matched set of antibodies, the method comprising:
(a) determining the affinity of each antibody for its respective antigen in a cell lysate,
(b) determining the selectivity of each antibody for its respective antigen in the cell lysate, and
(c) selecting antibodies to form the matched set,
wherein the affinity of the antibody and/or the selectivity of the antibody are determined using immunoprecipitation-mass spectrometry (IP-MS), and
wherein the matched set is composed of two or more antibodies that each have selectivity of at least 100 fold enrichment of for its respective antigen present in the cell lysate.

48. The method of claim 47, wherein the two or more antibodies that have affinities for their respective antigens with one log of each other.

49. The method of claim 47, wherein the matched set contains from three to ten antibodies.

50. The method of claim 47, wherein the antibodies bind to related targets.

51. The methods of claim 47, wherein the related targets are pre- and post-translationally modified forms of the same protein.

52. The methods of claim 51, wherein the pre-translationally modified form of the protein unphosphorylated and the post-translationally modified form of the protein phosphorylated.

53. A method for determining the selectivity of an antibody, the method comprising:
(a) contacting the antibody with a cell extract under conditions that allow for the formation on an immunoprecipitate between the antibody and one or more antigen in the cell extract,
(b) collecting the immunprecipitate formed in step (a), and
(c) indentifying one or more non-antibody molecules present in the immunprecipitate by mass spectrometry,
wherein the cell extract contains cell components from two or more cell types or one or more cell types from two or more species.

54. The method of claim 53, wherein the two or more cell types are from the same species.

55. The method of claim 53, wherein the cell types are obtained from two or more of the following tissues:
(a) muscular,
(b) connective,
(c) nervous, and
(d) epithelial.

56. The method of claim 53, wherein the connective tissue is blood.

57. The method of claim 53, wherein the two or more cell types are from different species.

58. The method of claim 57, wherein the two or more cell types from different species are obtained from two or more tissues from each species.

59. A method for determining the selectivity of an antibody, the method comprising:
(a) contacting the antibody with two or more proteins under conditions that allow for the formation on an immunoprecipitate between the antibody and one or more of the two or more proteins,
(b) collecting the immunprecipitate formed in step (a), and
(c) quantifying the amount of individual proteins present in the immunprecipitate by mass spectrometry.

60. A method for identifying an antibody that selectively binds to target molecules of cells obtained from different species, the method comprising:
(a) contacting the antibody with two or more cell lysates generated from cell of different species under conditions that allow for the formation on two or more immunoprecipitates between the antibody and one or more target molecule present in each cell lysate,
(b) collecting the immunoprecipitate from each cell lysate, and
(c) determining the fold purification for the target molecules in each immunoprecipitate by mass spectrometry.

61. The method of claim 65, wherein the species are selected from the group consisting of:
(a) Homo sapiens,
(b) Oryctolagus cuniculus,
(c) Mus musculus, and
(d) Rattus norvegicus.

62. The method of claim 65, wherein the antibody is generated in response to an epitope or a protein that is conserved across the different species from which the cell lysates are obtained.

63. The method of claim 67, wherein the epitope is from a protein or the protein is in a category selected from the group consisting of:
(a) heat shock proteins,
(b) polymerases,
(c) cell surface receptors,
(d) transcription factors,
(e) kinases,
(f) dephosphorylases,
(g) membrane associated transporters, and
(h) zinc finger proteins.


SEQUENCE LISTING (1 sequence)

SEQ ID NO: 1 - protein, 21 aa, Mus sp.
NAIFKALQADSPHSSSGMSEV